(19) United States
(12) Patent Application Publication
(10) Pub. No.: US 2003/0232868 A1
(43) Pub. Date: Dec. 18, 2003

(54) Cyclic carboxylic acids as integrin antagonists

(76) Inventors: Thomas Lehmann, Gladbach (DE); Rudiger Fischer, Pulheim (DE); Markus Albers, Leverkusen (DE); Thomas Rolle, Leverkusen (DE); Gerhard Muller, Krefeld (DE); Gerhard Hessler, Hofheim (DE); Masaomi Tajimi, Kyoto-fu (JP); Karl Ziegelbauer, Haan (DE); Hiromi Okigami, Kyoto-fu (JP); Kevin Bacon, Hyogo-ken (JP); Haruki Hasegawa, Ann Arbor, MI (US)

Correspondence Address: JEFFREY M. GREENMAN, VICE PRESIDENT, PATENTS AND LICENSING, BAYER CORPORATION, 400 MORGAN LANE, WEST HAVEN, CT, 06516, US

(21) Appl. No.: 10/258,079

(22) PCT Filed: Apr. 9, 2001
(86) PCT No.: PCT/EP01/04043

(30) Foreign Application Priority Data
Apr. 20, 2000 (DE) 10019755.8

(51) Int. Cl. A61K 31/41; A61K 31/195; C07D 249/16; C07C 275/38

(52) U.S. Cl. 514/381; 514/563; 548/257; 562/439

(57) ABSTRACT

The present invention relates to compounds of general formula (I), processes for their preparation, pharmaceutical compositions containing them as well as their use for the production of pharmaceutical compositions for the treatment of inflammatory diseases.

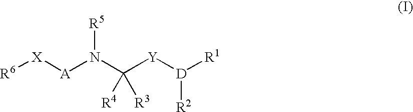

The present invention relates to compounds of formula (I),

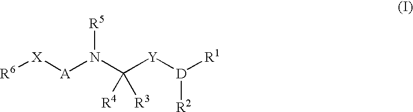

their preparation and use as pharmaceutical compositions as integrin antagonists, especially as α₄β₁ and/or α₄β₇ and/or α₉β₁ integrin antagonists and in particular for the production of pharmaceutical compositions suitable for the inhibition or the prevention of cell adhesion and cell-adhesion mediated disorders. Examples are the treatment and the prophylaxis of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

Adhesive interactions between the leukocytes and endothelial cells play a critical role in leukocyte trafficking to sites of inflammation. These events are essential for normal host defense against pathogens and repair of tissue damage, but can also contribute to the pathology of a variety of inflammatory and autoimmune disorders. Indeed, eosinophil and T cell infiltration into the tissue is known as a cardinal feature of allergic inflammation such as asthma.

The interaction of circulating leukocytes with adhesion molecules on the luminal surface of blood vessels appears to modulate leukocyte transmigration. These vascular cell adhesion molecules arrest circulating leukocytes, thereby serving as the first step in their recruitment to infected or inflamed tissue sites. Subsequently, the leukocytes reaching the extravascular space interact with connective tissue cells such as fibroblasts as well as extracellular matrix proteins such as fibronectin, laminin, and collagen. Adhesion molecules on the leukocytes and on the vascular endothelium are hence essential to leukocyte migration and attractive therapeutic targets for intervention in many inflammatory disorders.

Leukocyte recruitment to sites of inflammation occurs in a stepwise fashion beginning with leukocyte tethering to the endothelial cells lining the blood vessels. This is followed by leukocyte rolling, activation, firm adhesion, and transmigration. A number of cell adhesion molecules involved in those four recruitment steps have been identified and characterized to date. Among them, the interaction between vascular cell adhesion molecule 1 (VCAM-1) and very late antigen 4 (VLA-4, α₄⊕₁ integrin), as well as the interaction between mucosal addressin cell adhesion molecule 1 (MAdCAM-1) and α₄β₇ integrin, has been shown to mediate the tethering, rolling, and adhesion of lymphocytes and eosinophils, but not neutrophils, to endothelial cells under a physiologic flow condition. This suggests that the VCAM-1/VLA-4 and/or MAdCAM-1/α₄β₇ integrin mediated interactions could predominantly mediate a selective recruitment of leukocyte subpopulations in vivo. The inhibition of this interaction is a point of departure for therapeutic intervention (A. J. Wardlaw, J. Allergy Clin. Immunol. 1999, 104, 917-26).

VCAM-1 is a member of immunoglobulin (Ig) superfamily and is one of the key regulators of leukocyte trafficking to sites of inflammation. VCAM-1, along with intracellular adhesion molecule 1 (ICAM-1) and E-selectin, is expressed on inflamed endothelium activated by such cytolines as interleukin 1 (IL-1) and tumor necrosis factor a (TINF-α), as well as by lipopolysaccharide (LPS), via nuclear factor κB (NF-κB) dependent pathway. However, these molecules are not expressed on resting endothelium. Cell adhesion mediated by VCAM-1 may be involved in numerous physiological and pathological processes including myogenesis, hematopoiesis, inflammatory reactions, and the development of autoimmune disorders. Integins VLA-4 and α₄β₇ both function as leukocyte receptors for VCAM-1.

The integrin α₄β₁ is a heterodimeric protein expressed in substantial levels on all circulating leukocytes except mature neutrophils. It regulates cell migration into tissues during inflammatory responses and normal lymphocyte trafficking. VLA-4 binds to different primary sequence determinants, such as a QIDSP motif of VCAM-1 and an ILDVP sequence of the major cell type-specific adhesion site of the alternatively spliced type HI connecting segment domain (CS-1) of fibronectin.

In vivo studies with neutralizing monoclonal antibodies and inhibitor peptides have demonstrated a critical role for α₄ integrins interaction in leukocyte-mediated inflammation. Blocking of VLA-4/ligand interactions, thus, holds promise for therapeutic intervention in a variety of inflammatory, autoimmune and immune diseases (Zimmerman, C.; Exp. Opin. Ther. Patents 1999, 9, 129-133).

Furthermore, compounds containing a bisarylurea moiety as a substituent were disclosed as α₄β₁ integrin receptor antagonists: WO 96/22966, WO 97/03094, WO 99/33789, WO 99/37605. However, no aminobenzoic acids or aminocycloalkyl-carboxylic acids or homologues thereof or heterocyclics analogues thereof with α₄β₁ integrin receptor antagonists activity have been described.

3-[[[(phenylacetyl)amino]acetyl]amino]-benzoic acid has been described in Biochemistry, Vol. 26, No. 12, 1987, 3385 as a substrate for β-lactamases. N-(4-amino-phenylacetylglycyl)-4-aminophenylacetic acid has been described in J. für prakt. Chem., 4. Reihe, Band 27, 1965, 63 without giving a pharmaceutical use. N¹-[4-(ethoxycarbonyl)phenyl]-N²-(phenylacetyl)-α-glutamine and N²-benzoyl-N¹-[4-(ethoxycarbonyl)phenyl]-α-glutamine and related compounds have been described in Minerva Medica, 58 (86), 1967, 3651 and NL 6510006 as antisecretory agents. (S) 4-[[4-carboxy-1-oxo-2-[(phenylacetyl)amino]butyl]amino]-benzeneacetic acid has been described in Drugs Exp. Clin. Res. Suppl. 1, XIII, 1987, 57 as antitumor agent. N-[2-[[4-aminosulfonyl)phenylamino]-2-oxoethyl]-N-ethylbenzeneacetamide has been described in Eur. J. Med. Chem.-Chim. Ther. 12 (4), 1977, 387 with schistosomicide activity. N-(2-phenylacetylamino-acetylamino)-benzoic acid ethyl ester has been described in Yakugaku Zasshi 79, 1959, 1606 in decomposition studies of penicillins. Japanese publication Hei 11-269135 describes 3-aminosubstituted benzoic acid derivatives as selectin inhibitors.

None of these compounds have been described in relation to the inhibition or the prevention of cell adhesion and cell-adhesion mediated disorders.

Further to their α₄β₁ integrin antagonistic activity, the compounds of the present invention may also be used as α₄β₇ or α₉β₁ integin antagonists.

An object of the present invention is to provide new, alternative, aminobenzoic acids or aminocycloalkylcarboxylic acids or homologues thereof or heterocyclic analogues thereof derived integrin antagonists for the treatment of inflammatory, autoimmune and immune diseases.

The present invention therefore relates to compounds of the general formula (I):

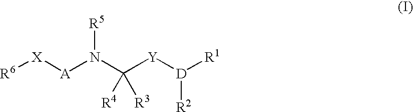

wherein

R¹ represents a 4- to 9-membered saturated, unsaturated or aromatic cyclic residue,

which can contain 0 to 3 heteroatoms selected independently from the group N, S and O,

wherein the cyclic residue R¹ can be annulated with a 4- to 8-membered saturated, unsaturated or aromatic cyclic residue, which can contain 0 to 2 heteroatoms selected independently from the group N, S and O,

and wherein the cyclic residue R¹ and/or a ring annulated to the cyclic residue R¹ is substituted by 1 to 2 substituents —R¹⁻¹—R¹⁻²—R¹⁻³-Z, wherein

R¹⁻¹ represents a bond, —O—, —S—, NR¹⁻⁴, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,

wherein R¹⁻¹ can optionally be substituted by 1 to 2 substituents selected from the group R¹⁻⁵,

wherein R¹⁻⁵ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,

wherein R¹⁻⁵ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,

R¹⁻² represents a bond, —O—, —S—, NR¹⁻⁴, C₁-C₁₀ alkyl, C₂-C₁₀ alkenyl, C₂-C₁₀ alkynyl,

wherein R¹⁻² can optionally be substituted by C₁-C₁₀ alkyl, C₂-C₁₀ alkenyl, C₂-C₁₀ alkynyl or R¹⁻⁶,

wherein R¹⁻⁶ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,

wherein R¹⁻⁶ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,

R¹⁻⁴ can optionally be hydrogen, C₁-C₁₀ alkyl, C₂-C₁₀ alkenyl or C₂-C₁₀ alkynyl,

R¹⁻³ represents a bond, C₁-C₁₀ alkyl, C₂-C₁₀ alkenyl, C₂-C₁₀ alkynyl,

wherein R¹⁻³ can optionally be substituted by C₁-C₁₀ alkyl, C₂-C₁₀ alkenyl, C₂-C₁₀ alkynyl or R¹⁻⁷,

wherein R¹⁻⁷ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,

wherein R¹⁻⁷ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,

with the proviso that, where R¹⁻³ is a bond, R¹⁻² is not a heteroatom,

and with the proviso that R¹⁻¹ and R¹⁻² are not both heteroatom at the same time,

Z represents —C(O)OR^{Z-1}, —C(O)NR^{Z-2}R^{Z-3}, —SO₂NR^{Z-2}R^{Z-3}, —SO(OR^{Z-1}), —SO₂(OR^{Z-1}), —P(O)R^{Z-1}(OR^{Z-3}), —PO(OR^{Z-1})(OR^{Z-3}) or 5-tetrazolyl,

wherein R^{Z-2} is hydrogen, C₁-C₄ alkyl, C₂-C₆ alkenyl, C₂-C₆ alkynyl, C₃-C₆ cycloalkyl, C₆ or C₁₀ aryl, —C(O)R^{Z-4} or —SO₂R^{Z-4},

wherein R^{Z-4} is C₁-C₄ alkyl, C₂-C₆ alkenyl, C₂-C₆ alkynyl, C₃-C₆ cycloalkyl, C₆ or C₁₀ aryl,

wherein R^{Z-4} can optionally be substituted by 1 to 3 substituents selected from the group halogen, nitro, cyano, oxo,

R^{Z-1} and R^{Z-3} are identical or different and represent hydrogen, C₁-C₄ alkyl, C₂-C₆ alkenyl, C₂-C₆ alkynyl, C₃-C₆ cycloalkyl, C₆ or C₁₀ aryl or benzyl,

wherein R^{Z-1} and R^{Z-3} can optionally be substituted by 1 to 3 substituents selected from the group C₁-C₄ alkyl, C₁-C₄ alkyloxy, halogen, nitro, cyano,

the cyclic residue R¹ and/or a ring annulated to the cyclic residue formed by R¹ can optionally be substituted by 0 to 2 substituents R¹⁻⁸, halogen, nitro, amino, cyano and oxo,

wherein

R¹⁻⁸ can independently be selected from the group of C₁-C₄ alkyl, C₁-C₄ alkyloxy, phenyl, phenoxy, phenylamino, C₃-C₆ cycloalkyl, and

R² represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,

which can optionally be substituted by 1 to 3 radicals R²⁻¹,

wherein R²⁻¹ represents C₁₋₄ alkyl, trifluormethyl, trifluormethoxy, —OR²⁻², —SR²⁻², NR²⁻³R²⁻⁴, —C(O)R²⁻², S(O)R²⁻², —SO₂R²⁻², —CO₂R²⁻², —OC(O)R²⁻², —C(O)NR²⁻³R²⁻⁴, —NR²⁻²C(O)R²⁻³, —SO₂NR²⁻³R²⁻⁴, NR²⁻²SO₂R²⁻³, —NR²⁻²C(O)NR²⁻³R²⁻⁴, NR²⁻²C(O)OR²⁻³, —OC(O)NR²⁻³R²⁻⁴, halogen, cyano, nitro or oxo,

wherein R²⁻² represents hydrogen, C₁-C₄ alkyl, C₃-C₆ cycloalkyl, C₆ or C₁₀ aryl

which can optionally be substituted by 1 substituent selected from the group C₁-C₄ alkyl, C₁-C₄ alkyloxy, phenyl, C₃-C₆ cycloalkyl, halogen, nitro, cyano,

and wherein R²⁻³ and R²⁻⁴ are identical or different and represent hydrogen, C₁-C₄ alkyl, C₃-C₆ cycloalkyl, C₆ or C₁₀ aryl, or

R²⁻³ and R²⁻⁴ together form a 4-7-membered ring, which includes the nitrogen atom to which R²⁻³ and R²⁻⁴ are bonded and which contains up to 2 additional heteroatoms selected from the group oxygen, nitrogen or sulfur and which contains up to 2 double bonds,

and if R² is alkyl, R² together with the cyclic residue R¹ and D can form a ring,

R³ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,

wherein R³ can optionally be substituted by 1 to 3 radicals R³⁻¹,

and wherein R³ can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur, which can be annulated with a phenyl ring,

and which can optionally be substituted by 1 to 3 radicals R³⁻¹,

wherein R³⁻¹ represents C₁-C₄ alkyl, trifluormethyl, trifluormethoxy, —OR³⁻², —SR³⁻², NR³⁻³R³⁻⁴, —C(O)R³⁻², S(O)R³⁻², —SO₂R³⁻², —OC(O)R³⁻², —C(O)NR³⁻³R³⁻⁴, —NR³⁻²C(O)R³⁻³, —SO₂NR³⁻⁴, NR³⁻²SO₂R³⁻³, —NR³⁻²C(O)NR³⁻³R³⁻⁴, —NR³⁻²C(O)OR³⁻³, —OC(O)NR³⁻³R³⁻⁴, —CO₂R³⁻⁵, halogen, cyano, nitro or oxo,

wherein R³⁻² represents hydrogen, C₁-C₄ alkyl, C₃-C₆ cycloalkyl, C₆ or C₁₀ aryl

which can optionally be substituted by 1 substituent selected from the group C₁-C₄ alkyl, C₁-C₄ alkyloxy, phenyl, C₃-C₆ cycloalkyl, halogen, nitro, cyano,

and wherein R³⁻³ and R³⁻⁴ are identical or different and represent hydrogen, C₁-C₄ alkyl, C₃-C₆ cycloalkyl, C₆ or C₁₀ aryl, benzyl or 9-fluorenylmethyl, or

R³⁻³ and R³⁻⁴ together, form a 4-7-membered ring, which includes the nitrogen atom to which R³⁻³ and R³⁻⁴ are bonded and which contains up to 2 additional heteroatoms selected from the group oxygen, nitrogen or sulfur and which contains up to 2 double bonds,

and wherein R³⁻⁵ represents C₁-C₄ alkyl, C₃-C₆ cycloalkyl, C₆ or C₁₀ aryl

R⁴ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,

which can optionally be substituted by 1 to 3 radicals R⁴⁻¹,

and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,

which can optionally be substituted by 1 to 3 radicals R⁴⁻¹,

wherein R⁴⁻¹ represents C₁-C₄ alkyl, trifluormethyl, trifluormethoxy, —OR⁴⁻², —SR⁴⁻², NR⁴⁻³R⁴⁻⁴, —C(O)R⁴⁻², S(O)R⁴⁻², —SO₂R⁴⁻², —OC(O)R⁴⁻², —C(O)NR⁴⁻³R⁴⁻⁴, —NR⁴⁻²C(O)R⁴⁻³, —SO₂NR⁴⁻³R⁴⁻⁴, NR⁴⁻²SO₂R⁴⁻³, —NR⁴⁻²C(O)NR⁴⁻⁴, —NR⁴⁻²C(O)OR⁴⁻³, —OC(O)NR⁴⁻³R⁴⁻⁴, —CO₂R⁴⁻⁵, halogen, cyano, nitro or oxo,

wherein R⁴⁻² represents hydrogen, C₁-C₄ alkyl, C₃-C₆ cycloalkyl, C₆ or C₁₀ aryl

which can optionally be substituted by 1 substituent selected from the group C₁-C₄ alkyl, C₁-C₄ alkyloxy, phenyl, C₃-C₆ cycloalkyl, halogen, nitro, cyano,

and wherein R⁴⁻³ and R⁴⁻⁴ are identical or different and represent hydrogen, C₁₋₄ alkyl, C₃-C₆ cycloalkyl, C₆ or C₁₀ aryl, or

R⁴⁻³ and R⁴⁻⁴ together form a 4-7-membered ring, which includes the nitrogen atom to which R⁴⁻³ and R⁴⁻⁴ are bonded and which contains up to 2 additional heteroatoms selected from the group oxygen, nitrogen or sulfur and which contains up to 2 double bonds,

and wherein R⁴⁻⁵ represents C₁-C₄ alkyl, C₃-C₆ cycloalkyl, C₆ or C₁₀ aryl

R⁵ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,

which can optionally be substituted by 1 to 3 radicals R⁵⁻¹,

and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,

which can optionally be substituted by 1 to 3 radicals R⁵⁻¹,

wherein R⁵⁻¹ represents C₁-C₄ alkyl, trifluormethyl, trifluormethoxy, —OR⁵⁻², —SR⁵⁻², NR⁵⁻³R⁵⁻⁴, C(O)R⁵⁻², S(O)R⁵⁻²SO₂R⁵⁻², —CO₂R⁵⁻², —OC(O)R⁵⁻², —C(O)NR⁵⁻³R⁵⁻⁴, —NR⁵⁻²C(O)R⁵⁻³, —SO₂NR⁵⁻³R⁵⁻⁴, NR⁵⁻²SO₂R⁵⁻³, —NR⁵⁻²C(O)NR⁵⁻³R⁵⁻⁴, —NR⁵⁻²C(O)OR⁵⁻³, —OC(O)NR⁵⁻³R⁵⁻⁴, halogen, cyano, nitro or oxo,

wherein R⁵⁻² represents hydrogen, C₁-C₄ alkyl, C₃-C₆ cycloalkyl, C₆ or C₁₀ aryl

which can optionally be substituted by 1 substituent selected from the group C₁-C₄ alkyl, C₁-C₄ alkyloxy, phenyl, C₃-C₆ cycloalkyl, halogen, nitro, cyano,

and wherein R⁵⁻³ and R⁵⁻⁴ are identical or different and represent hydrogen, C₁-C₄ alkyl, C₃-C₆ cycloalkyl, C₆ or C₁₀ aryl, or

R⁵⁻³ and R⁵⁻⁴ together form a 4-7-membered ring, which includes the nitrogen atom to which R⁵⁻³ and R⁵⁻⁴ are bonded and which contains up to 2 additional heteroatoms selected from the group oxygen, nitrogen or sulfur and which contains up to 2 double bonds,

R⁶ represents phenyl or a 5- to 6-membered aromatic heterocyclic residue containing up to 3 heteroatoms independently selected from the group oxygen, nitrogen or sulfur,

which can optionally be annulated with a 5- to 8-membered saturated or unsaturated cyclic residue containing up to 2 heteroatoms independently selected from the group oxygen, nitrogen or sulfur,

and which can optionally be independently substituted by 1 to 3 radicals R⁶⁻¹ and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,

wherein the latter cyclic substituents can themselves optionally be substituted by 1 to 3 radicals R⁶⁻¹,

wherein R⁶⁻¹ represents C₁-C₄ alkyl, trifluormethyl, trifluoromethoxy, —OR⁶⁻⁴, —SR⁶⁻², NR⁶⁻³R⁶⁻⁴, —C(O)R⁶⁻², S(O)R⁶⁻², —SO₂R⁶⁻², —CO₂R⁶⁻², —OC(O)R⁶⁻², —C(O)NR⁶⁻³R⁶⁻⁴, —NR⁶⁻²C(O)R⁶⁻², —SO₂NR⁶⁻³R⁶⁻⁴, —NR⁶⁻²SO₂R⁶⁻², —NR⁶⁻²C(O)NR⁶⁻³R⁶⁻⁴, —NR⁶⁻²C(O)OR⁶⁻⁴, —OC(O)NR⁶⁻³R⁶⁻⁴, halogen, cyano, nitro or oxo,

wherein R⁶⁻² represents hydrogen, C₁-C₄ alkyl, C₃-C₆ cycloalkyl, C₆ or C₁₀ aryl

which can optionally be substituted by 1 to 3 substituents selected from the group C₁-C₄ alkyl, C₁-C₄ alkyloxy, phenyl, C₃-C₆ cycloalkyl, halogen, nitro, cyano,

and wherein R⁶⁻³ and R⁶⁻⁴ are identical or different and represent hydrogen, C₁-C₄ alkyl, C₃-C₆ cycloalkyl, C₆ or C₁₀ aryl or a 4-9-membered saturated or unsaturated heterocyclic residue containing up to 2 heteroatoms selected from the group oxygen, nitrogen or sulfur,

which can optionally be substituted by 1 to 2 substituents selected from the group C₁-C₄ alkyl, phenyl, C₃-C₇ cycloalkyl, C₁-C₄ alkyloxy, halogen, nitro, cyano, or

R⁶⁻³ and R⁶⁻⁴ together form a 4-7-membered ring, which includes the nitrogen atom to which R⁶⁻³ and R⁶⁻⁴ are bonded and which contains up to 2 additional heteroatoms selected from the group oxygen, nitrogen or sulfur and which contains up to 2 double bonds, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo,

and in case that R¹ represents a 3-amino benzoic acid derivative and R⁶⁻¹ represents —OR⁶⁻⁴, —C(O)NR⁶⁻³R⁶⁻⁴ or —NR⁶⁻²C(O)R⁶⁻⁴, then R⁶⁻⁴ represents C₆ or C₁₀ aryl or a 4-9-membered saturated or unsaturated heterocyclic residue containing up to 2 heteroatoms selected from the group oxygen, nitrogen or sulfur,

wherein the ring formed by R⁶⁻³ and R⁶⁻⁴ can optionally be substituted by 1 to 2 substituents selected from the group C₁-C₄ alkyl, phenyl, C₃-C₇ cycloalkyl, C₁-C₄ alkyloxy, halogen, nitro, cyano, or

R³ and R⁴ or R⁴ and R⁵ together form a 4-7-membered saturated or unsaturated ring containing up to 2 heteroatoms selected from the group oxygen, nitrogen or sulfur, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo and which can be fused with a 3-7 membered homocyclic or heterocyclic, saturated, unsaturated or aromatic ring,

A represents —C(O)—, —C(O)—C(O)—, —SO—, —SO₂—, —PO—, —PO₂—, 2-pyrimidyl, 4-pyrimidyl, 2-pyridyl, 2-imidazolyl, 4-imidazolyl, 2-benzimidazolyl or a ring selected from the following group:

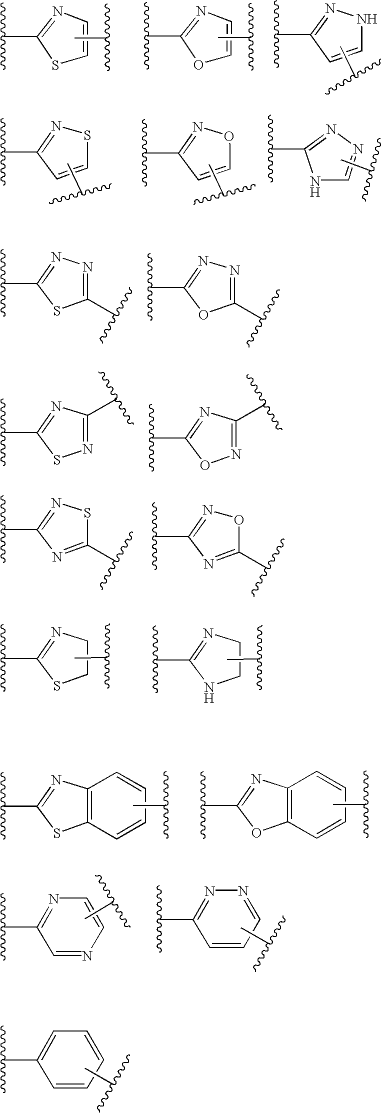

wherein the abovementioned ring systems can optionally be substituted by C₁-C₄ alkyl, C₁-C₄ alkoxy, halogen, nitro, amino, cyano,

X represents —CR^{X-1}R^{X-2},

wherein R^{X-1} and R^{X-2} can be independently selected from the group hydrogen, C₁-C₄ alkyl, C₂-C₄ alkenyl, C₂-C₄ alkynyl, or

together with R⁶ form a 4-7-membered ring, which can contain up to 2 heteroatoms independently selected from the group oxygen, nitrogen or sulfur and containing up to 2 double bonds, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo,

Y represents bond, —C(O)—, —S(O)—, —SO₂—, —O—, —S—, —CR^{Y-1}R^{Y-2}—, or —NR^{Y-3},

wherein R^{Y-1}, R^{Y-2}, R^{Y-3} can be independently selected from the group bond, hydrogen, C₁-C₄ alkyl, C₂-C₄ alkenyl, C₂-C₄ alkynyl,

and can optionally be substituted by 1 to 2 substituents independently selected from the group C₁-C₄ alkyl, phenyl, benzyl, C₃-C₇ cycloalkyl, C₁-C₄ alkyloxy, halogen, nitro, cyano, oxo,

D represents N or CR^{D-1},

wherein R^{D-1} can be independently selected from the group bond, hydrogen, C₁-C₄ alkyl, C₂-C₄ alkenyl, C₂-C₄ alkynyl,

and R^{D-1} can optionally be substituted by 1 to 2 substituents independently selected from the group C₁-C₄ alkyl, phenyl, benzyl, C₃-C₇ cycloalkyl, C₁-C₄ alkyloxy, halogen, nitro, cyano, oxo,

with the proviso that, where D represents —N—, Y does not represent —O— or —S—,

and the compound is not one of the following: 3-[[[(phenylacetyl)amino]acetyl]amino]-benzoic acid; N-(4-aminophenylacetylglycyl)-4-aminophenylacetic acid; N¹-[4-ethoxycarbonyl)phenyl]-N²-(phenylacetyl)-α-glutamine; N²-benzoyl-N¹-[4-(ethoxycarbonyl)phenyl]-α-glutamine; (S)-4-[[4-carboxy-1-oxo-2-[(phenylacetyl)amino]butyl]amino]-benzeneacetic acid; N-[2-[[4-aminosulfonyl)phenyl]amino]-2-oxoethyl]-N-ethylbenzeneacetamide; N-(2-phenylacetylamino-acetylamino)-benzoic acid ethyl ester,

and pharmaceutically acceptable salts thereof.

In a preferred embodiment, the present invention relates to compounds of general formula (I),

wherein

R¹ represents a 4 to 6-membered saturated, unsaturated or aromatic cyclic residue,

which can contain 0 to 3 heteroatoms selected independently from the group N, S and O,

wherein the cyclic residue R¹ can be annulated with a 5- to 6-membered saturated, unsaturated or aromatic cyclic residue, which can contain 0 to 2 heteroatoms selected independently from the group N, S and O,

and wherein the cyclic residue R¹ and/or a ring annulated to the cyclic residue R¹ is substituted by 1 to 2 substituents —R¹⁻—R¹⁻²—R¹⁻³-Z, wherein

R¹⁻¹ represents a bond, C₁-C₆ alkyl, C₂-C₆ alkenyl, C₂-C₆ alkynyl or C₆ aryl,

wherein R¹⁻¹ can optionally be substituted by 1 substituent selected from the group R¹⁻⁵, wherein R¹⁻⁵ represents hydrogen, C₁-C₆ alkyl, C₂-C₆ alkenyl, C₂-C₆ alkynyl, C₃-C₇ cycloalkyl or C₆ aryl,

R¹⁻² represents a bond, C₁-C₆ alkyl, C₂-C₆ alkenyl, C₂-C₆ alkynyl

R¹⁻³ represents a bond, C₁-C₆ alkyl, C₂-C₆ alkenyl, C₂-C₆ alkynyl

Z represents —C(O)OR^{Z-1}, —C(O)NR^{Z-2}R^{Z-3} or 5-tetrazolyl,

wherein R^{Z-1}, R^{Z-2} and R^{Z-3} are identical or different and represent hydrogen, C₁-C₄ alkyl, C₂-C₆ alkenyl, C₂-C₆ alkynyl or benzyl,

the cyclic residue R¹ and/or a ring annulated to the cyclic residue formed by R¹ can optionally be substituted by 0 to 2 substituents R¹⁻⁸, halogen, nitro, amino, cyano and oxo,

wherein

R¹⁻⁸ can independently be selected from the group of C₁-C₄ alkyl, C₁-C₄ alkyloxy, phenyl, phenoxy, phenylamino,

R² represents hydrogen, C₁-C₆ alkyl, C₂-C₆ alkenyl, C₂-C₆ alkynyl, C₆ aryl, C₅-C₆ cycloalkyl,

and if R² is alkyl, R² together with the cyclic residue R¹ and D can form a 5- to 6-membered ring,

R³ represents hydrogen, C₁-C₆ alkyl, C₂-C₆ alkenyl, C₂-C₆ alkynyl, C₆ aryl, C₅-C₆ cycloalkyl or a 5-6-membered saturated or unsaturated heterocyclic residue containing up to 2 heteroatoms selected from the group oxygen, nitrogen or sulfur,

which can optionally be substituted by 1 radical R³⁻¹,

and wherein R³ can furthermore be single-foldedly substituted by C₃-C₇ cycloalkyl, C₆ aryl, C₄-C₉ heteroaryl or a heterocyclic residue containing up to 2 heteroatoms selected from the group oxygen, nitrogen or sulfur, which can be annulated with a phenyl ring,

wherein R³⁻¹ represents trifluormethyl, trifluormethoxy, —OR³⁻², —SR³⁻², NR³⁻³R³⁻⁴, —NR³⁻²C(O)OR³⁻³, —CO₂R³⁻⁵, halogen, cyano, nitro or oxo,

wherein R³⁻² represents hydrogen or C₁-C₄ alkyl,

and wherein R³⁻³ and R³⁻⁴ are identical or different and represent hydrogen, C₁-C₄ alkyl or benzyl or 9-fluorenylmethyl,

and wherein R³⁻⁵ represents C₁-C₄ alkyl,

R⁴ represents hydrogen, C₁-C₆ alkyl, C₂-C₆ alkenyl, C₂-C₆ or C₆ aryl,

R⁵ represents hydrogen, C₁-C₆ alkyl, C₂-C₆ alkenyl, C₂-C₆ alkynyl or C₆ aryl,

which can optionally be substituted by 1 radical R⁵⁻¹,

wherein R⁵⁻¹ represents trifluormethyl, trifluormethoxy, —OR⁵⁻², —SR⁵⁻², NR⁵⁻³R⁵⁻⁴, halogen, cyano, nitro or oxo,

wherein R⁵⁻², R⁵⁻³ and R⁵⁻⁴ are identical or different and represent hydrogen or C₁-C₄ alkyl,

R⁶ represents phenyl or a 5- to 6-membered aromatic heterocyclic residue containing up to 3 heteroatoms independently selected from the group oxygen, nitrogen or sulfur,

and which can optionally be independently substituted by 1 to 3 radicals R⁶⁻¹

wherein R⁶⁻¹ represents —NR⁶⁻²C(O)NR⁶⁻³R⁶⁻⁴,

wherein R⁶⁻² and R⁶⁻³ are identical or different and represent hydrogen or C₁-C₄ alkyl,

and wherein R⁶⁻⁴ represents C₆ aryl,

which can optionally be substituted by 1-2 substituents selected from the group C₁-C₄ alkyl, C₁-C₄ alkyloxy, halogen, nitro, cyano,

or R³ and R⁴ or R⁴ and R⁵ together form a 5-6-membered saturated or unsaturated ring containing up to 2 nitrogen atoms,

A represents —C(O)—, —SO—, —SO₂—,

X represents —CR^{X-1}R^{X-2},

wherein R^{X-1} and R^{X-2} can be independently selected from the group hydrogen, C₁-C₄ alkyl,

Y represents —C(O)—,

D represents —N—,

and pharmaceutically acceptable salts thereof.

In another preferred embodiment, the present invention relates to compounds of general formula (I),

wherein

R¹ represents a 5- to 6-membered saturated, unsaturated or aromatic cyclic residue,

which can contain 0 to 3 heteroatoms selected independently from the group N and S,

wherein the cyclic residue R¹ can be annulated with a 5-membered unsaturated or aromatic cyclic residue, which contains 1 nitrogen atom,

and wherein the cyclic residue R¹ and/or a ring annulated to the cyclic residue R¹ is substituted by 1 to 2 substituents —R¹⁻¹—R¹⁻²—R¹⁻³-Z, wherein

R¹⁻¹ represents a bond or C₁ alkyl,

wherein R¹⁻¹ can optionally be substituted by cyclopentyl,

R¹⁻² represents a bond,

R¹⁻³ represents a bond,

Z represents —C(O)OR^{Z-1} or 5-tetrazolyl,

R^{Z-1} represents hydrogen, C₁-C₂ alkyl or benzyl,

the cyclic residue R¹ can optionally be substituted by 0 to 2 substituents R¹⁻⁸, halogen and nitro,

wherein

R¹⁻⁸ can independently be selected from the group of C₁-C₄ alkyloxy, phenoxy and phenylamino,

R² represents hydrogen or C₁-C₃ alkyl, or

and if R² is alkyl, R² together with the cyclic residue R¹ and D can form a piperidine ring,

R³ represents hydrogen or C₁-C₄ alkyl,

which can optionally be substituted by 1 radical R³⁻¹,

wherein R³⁻¹ represents NR³⁻³R³⁻⁴ or —NR³⁻²C(O)OR³⁻³,

wherein R³⁻² and R³⁻⁴ represent hydrogen,

R³⁻³ represents hydrogen, benzyl or 9-fluorenylmethyl,

R⁴ represents hydrogen,

R⁵ represents hydrogen or C₃ alkyl,

which can optionally be substituted by 1 radical R⁵⁻¹,

wherein R⁵⁻¹ represents —OR⁵⁻²,

wherein R⁵⁻² represents C₁ alkyl,

R⁶ represents phenyl,

and which is substituted by 1 radical R⁶⁻¹

wherein R⁶⁻¹ represents —NR⁶⁻²C(O)NR⁶⁻³R⁶⁻⁴,

wherein R⁶⁻² represents hydrogen,

and wherein R⁶⁻³ represents hydrogen

and R⁶⁻⁴ represents C₆ aryl,

which is substituted by 1 substituent C₁ alkyl,

A represents —C(O)—,

X represents —CR^{X-1}R^{X-2}—,

wherein R^{X-1} and R^{X-2} represent hydrogen,

Y represents —C(O)—,

D represents N,

and pharmaceutically acceptable salts thereof

In another preferred embodiment, the present invention relates to compounds of general formula (I),

wherein

R¹ represents phenyl,

and wherein the phenyl is substituted by 1 to 2 substituents —R¹⁻¹—R¹⁻²—R¹⁻³-Z,

wherein

R¹⁻¹ represents a bond or C₁ alkyl,

R¹⁻² represents a bond,

R¹⁻³ represents a bond,

In another preferred embodiment, the present invention relates to compounds of general formula (I), wherein

Z represents —C(O)OR^{Z-1}

R^{Z-1} represents hydrogen, C₁-C₂ alkyl or benzyl,

R² represents hydrogen,

R³ represents hydrogen, C₁-C₆ alkyl, C₂-C₆ alkenyl, C₂-C₆ alkynyl, C₆ aryl, C₅-C₆ cycloalkyl or a 5-6-membered saturated or unsaturated heterocyclic residue containing up to 2 heteroatoms selected from the group oxygen, nitrogen or sulfur,

which can optionally be substituted by 1 radical R³⁻¹,

and wherein R³ can furthermore be single-foldedly substituted by C₃-C₇ cycloalkyl C₆ aryl, C₄-C₉ heteroaryl or a heterocyclic residue containing up to 2 heteroatoms selected from the group oxygen, nitrogen or sulfur, which can be annulated with a phenyl ring,

wherein R³⁻¹ represents trifluormethyl, trifluormethoxy, —OR³⁻², —SR³⁻², —NR³⁻³R³⁻⁴, —NR³⁻²C(O)OR³⁻³, —CO₂R³⁻⁵, halogen, cyano, nitro or oxo,

wherein R³⁻² represents hydrogen or C₁-C₄ alkyl,

and wherein R³⁻³ and R³⁻⁴ are identical or different and represent hydrogen, C₁-C₄ alkyl or benzyl or 9-fluorenylmethyl,

and wherein R³⁻⁵ represents C₁-C₄ alkyl,

R⁴ represents hydrogen,

R⁵ represents hydrogen,

R⁶ represents phenyl,

and which is substituted by 1 radical R⁶⁻¹

wherein R⁶⁻¹ represents —NR⁶⁻²C(O)NR⁶⁻³R⁶⁻⁴,

wherein R⁶⁻² represents hydrogen,

and wherein R⁶⁻³ represents hydrogen

and R⁶⁻⁴ represents C₆ aryl,

which is substituted by 1 substituent C₁ alkyl,

or R³ and R⁴ or R⁴ and R⁵ together form a 5-6-membered saturated or a unsaturated ring containing up to 2 nitrogen atoms,

A represents —C(O)—,

X represents —CR^{X-1}R^{X-2}—,

wherein R^{X-1} and R^{X-2} represent hydrogen,

Y represents —C(O)—,

D represents N,

and pharmaceutically acceptable salts thereof.

In a more preferred embodiment, the present invention relates to compounds of general formula (I),

wherein

R¹ represents phenyl,

which is 1,4-substituted by a substituent —R¹⁻¹—R¹⁻²—R¹⁻³-Z,

wherein

R¹⁻¹, R¹⁻² and R¹⁻³ represent bonds.

In another more preferred embodiment, the present invention relates to compounds of general formula (I),

wherein

R¹ represents phenyl,

which is 1,3-substituted by a substituent —R¹⁻¹—R¹⁻²—R¹⁻³-Z,

wherein

R¹⁻¹ represents —CH₂—,

R¹⁻² and R¹⁻³ represent bonds.

In another more preferred embodiment, the present invention relates to compounds of general formula (I),

wherein

R¹ represents a 5-membered heterocycle.

In another more preferred embodiment, the present invention relates to compounds of general formula (I),

wherein

R¹ represents a cyclohexyl ring.

In a very preferred embodiment, the present invention relates to compounds of general formula (I),

wherein R⁶ represents

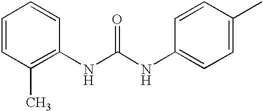

A preferred process for preparation of compounds of general formula (VII)

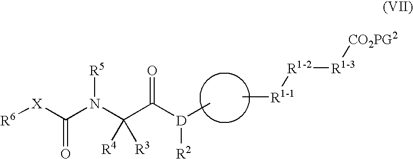

has also been found, which comprises reaction of carboxylic acids of general formula (V)

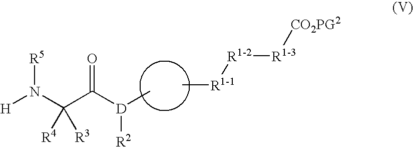

or activated derivatives thereof,

with compounds of the general formula (VI)

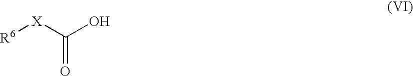

in the presence of a coupling agent and a base in inert solvents, which will be described in more detail in the descriptive part of the specification.

In the context of the present invention alkyl stands for a straight-chain or branched alkyl residue, such as methyl, ethyl, n-propyl, iso-propyl, n-pentyl. If not stated otherwise, preferred is C₁-C₁₀ alkyl, very preferred is C₁-C₆ alkyl.

Alkenyl and alkinyl stand for straight-chain or branched residues containing one or more double or triple bonds, e.g. vinyl, allyl, isopropinyl, ethinyl. If not stated otherwise, preferred is C₁-C₁₀ alkenyl or alkinyl, very preferred is C₁-C₆ alkenyl or alkinyl.

Cycloalkyl stands for a cyclic alkyl group such as cyclopropyl, cyclobutyl, cyclopentyl, cyclohexyl or cycloheptyl. Preferred is C₃-C₇ cycloalkyl.

Halogen in the context of the present invention stands for fluorine, chlorine, bromine or iodine. If not specified otherwise, chlorine or fluorine are preferred.

Heteroaryl stands for a monocyclic heteroaromatic system containing 4 to 9 ring atoms, which can be attached via a carbon atom or eventually via a nitrogen atom within the ring, for example, furan-2-yl, furan-3-yl, pyrrol-1-yl, pyrrol-2-yl, pyrrol-3-yl, thienyl, thiazolyl, oxazolyl, imidazolyl, triazolyl, tetrazolyl, pyridyl, pyrinidyl or pyridazinyl. C₄-C₉ heteroaryl also stands for a 4 to 9-membered ring, wherein one or more of the carbon atoms are replaced by heteroatoms.

A saturated or unsaturated heterocyclic residue stands for a heterocyclic system containing 4 to 9 ring atoms, which can contain one or more double bonds and which can be attached via a ring carbon atom or eventually via a nitrogen atom, e.g. tetrahydrofur-2-yl, pyrrolidine-1-yl, piperidine-1-yl, piperidine-2-yl, piperidine-3-yl, piperidine-4-yl, piperazine-1-yl, piperazine-2-yl morpholine-1-yl, 1,4-diazepine-1-yl or 1,4-dihydropyridine-1-yl.

If not specified otherwise, in the context of the present invention heteroatom stands preferably for O, S, N or P.

Annulated describes 1,1- or 1,2-fused ring systems, e.g. spiro systems or systems with a [0]-bridge. If not stated otherwise, substituents described for the “parent” ring system (the ring to which the annulated ring is attached) can be also present on the annulated ring.

Derivative stands for a compound that is derived from the parent compound by exchange of one or more hydrogen atoms by other functional groups.

Surprisingly, the compounds of the present invention show good integrin antagonistic activity. They are therefore suitable especially as α₄β₁ and/or α₄β₇ and/or α₉β₁ integrin antagonists and in particular for the production of pharmaceutical compositions for the inhibition or the prevention of cell adhesion and cell-adhesion mediated disorders. Examples are the treatment and the prophylaxis of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

The integrin antagonists of the invention are useful not only for treatment of the physiological conditions discussed above, but are also useful in such activities as purification of integrins and testing for activity.

For the treatment of the above-mentioned diseases, the compounds according to the invention can exhibit non-systemic or systemic activity, wherein the latter is preferred. To obtain systemic activity the active compounds can be administered, among other things, orally or parenterally, wherein oral administration is preferred.

For parenteral administration, forms of administration to the mucous membranes (i.e. buccal, lingual, sublingual, rectal, nasal, pulmonary, conjunctival or intravaginal) or into the interior of the body are particularly suitable. Administration can be carried out by avoiding absorption (i.e. intracardiac, intra-arterial, intravenous, intraspinal or intralumbar administration) or by including absorption (i.e. intracutaneous, subcutaneous, percutaneous, intramuscular or intraperitoneal administration).

For the above purpose the active compounds can be administered per se or in administration forms.

Suitable administration forms for oral administration are, inter alia, normal and enteric-coated tablets, capsules, coated tablets, pills, granules, pellets, powders, solid and liquid aerosols, syrups, emulsions, suspensions and solutions. Suitable administration forms for parenteral administration are injection and infusion solutions.

The active compound can be present in the administration forms in concentrations of from 0.001-100% by weight; preferably the concentration of the active compound should be 0.5-90% by weight, i.e. quantities which are sufficient to allow the specified range of dosage.

The active compounds can be converted in the known manner into the abovementioned administration forms using inert non-toxic pharmaceutically suitable auxiliaries, such as for example excipients, solvents, vehicles, emulsifiers and/or dispersants.

The following auxiliaries can be mentioned as examples: water, solid excipients such as ground natural or synthetic minerals (e.g. talcum or silicates), sugar (e.g. lactose), non-toxic organic solvents such as paraffins, vegetable oils (e.g. sesame oil), alcohols (e.g. ethanol, glycerol), glycols (e.g. polyethylene glycol), emulsifying agents, dispersants (e.g. polyvinylpyrrolidone) and lubricants (e.g. magnesium sulphate).

In the case of oral administration tablets can of course also contain additives such as sodium citrate as well as additives such as starch, gelatin and the like. Flavour enhancers or colorants can also be added to aqueous preparations for oral administration.

For the obtainment of effective results in the case of parenteral administration it has generally proven advantageous to administer quantities of about 0.001 to 100 mg/kg, preferably about 0.01 to 1 mg/kg of body weight In the case of oral administration the quantity is about 0.01 to 100 mg/kg, preferably about 0.1 to 10 mg/kg of body weight.

It may nevertheless be necessary to use quantities other than those mentioned above, depending on the body weight concerned, the method of administration, the individual response to the active compound, the type of preparation and the time or interval of administration.

Suitable pharmaceutically acceptable salts of the compounds of the present invention that contain an acidic moiety include addition salts formed with organic or inorganic bases. The salt forming ion derived from such bases can be metal ions, e.g., aluminum, alkali metal ions, such as sodium of potassium, alkaline earth metal ions such as calcium or magnesium, or an amine salt ion, of which a number are known for this purpose. Examples include ammonium salts, arylalkylamines such as dibenzylamine and N,N-dibenzylethylenediamine, lower alkylamines such as methylamine, t-butylamine, procaine, lower alkylpiperidines such as N-ethylpiperidine, cycloalkylamines such as cyclohexylamine or dicyclohexylamine, 1-adamantylamine, benzathine, or salts derived from amino acids like arginine, lysine or the like. The physiologically acceptable salts such as the sodium or potassium salts and the amino acid salts can be used medicinally as described above and are preferred.

Suitable pharmaceutically acceptable salts of the compounds of the present invention that contain a basic moiety include addition salts formed with organic or inorganic acids. The salt forming ion derived from such acids can be halide ions or ions of natural or unnatural carboxylic or sulfonic acids, of which a number are known for this purpose. Examples include chlorides, acetates, trifluoroacetates, tartrates, or salts derived from amino acids like glycine or the like. The physiologically acceptable salts such as the chloride salts, the trifluoroacetic acid salts and the amino acid salts can be used medicinally as described below and are preferred.

These and other salts which are not necessarily physiologically acceptable are useful in isolating or purifying a product acceptable for the purposes described below.

The salts are produced by reacting the acid form of the invention compound with an equivalent of the base supplying the desired basic ion or the basic form of the invention compound with an equivalent of the acid supplying the desired acid ion in a medium in which the salt precipitates or in aqueous medium and then lyophilizing. The free acid or basic form of the invention compounds can be obtained from the salt by conventional neutralization techniques, e.g., with potassium bisulfate, hydrochloric acid, sodium hydroxide, sodium bicarbonate, etc.

The compounds according to the invention can form non covalent addition compounds such as adducts or inclusion compounds like hydrates or clathrates. This is known to the artisan and such compounds are also object of the present invention.

The compounds according to the invention can exist in different stereoisomeric forms, which relate to each other in an enantiomeric way (image and mirror image) or in a diastereomeric way (image different from mirror image). The invention relates to the enantiomers and the diastereomers as well as their mixtures. They can be separated according to customary methods.

The compounds according to the invention can exist in tautomeric forms. This is known to the artisan and such compounds are also object of the present invention.

General Compound Synthesis

The synthesis of compounds according to the general formula (I) can be illustrated by the following scheme 1:

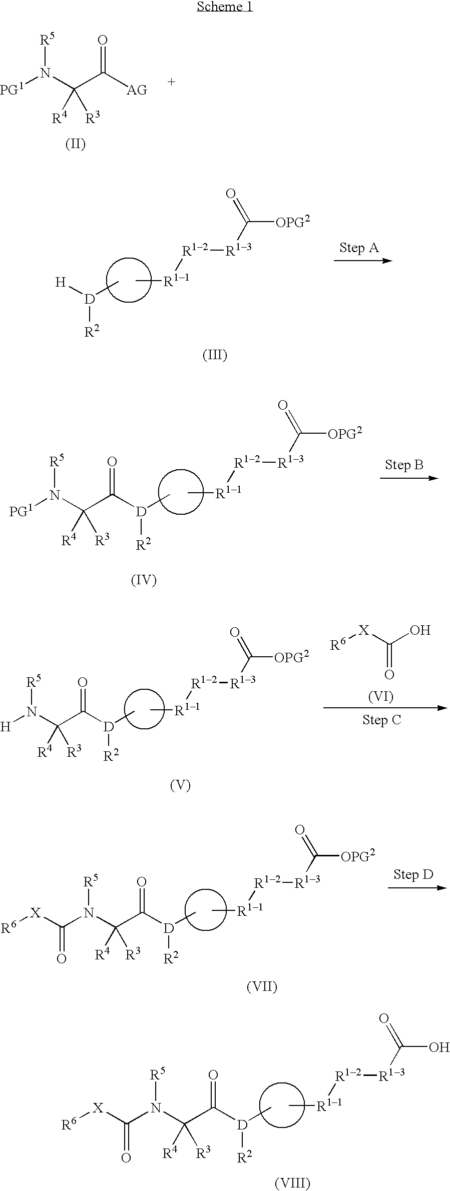

By coupling of the carboxylic acids or activated derivatives (II) with the amines (m) (D=nitrogen), followed by removal of the protecting group PG¹ the amides (V) can be obtained. Coupling with the carboxylic acids (VI) followed by removal of the protecting group PG² affords carboxylic acids of type (VIII). Further examples with different A, Y and D groups as defined in formula (I) are described below.

In the above scheme the depicted ring in formulas (III)-(V), (VII) and (VIII) as well as in scheme 3 represents a cyclic moiety formed by R¹. AG stands for hydroxyl or a suitable activating group forming an activated carboxylic acid derivative. Activated carboxylic acids derivatives of this type are known to the person skilled in the art and are described in detail in standard textbooks such as, for example in (i) Houben-Weyl, Methoden der organischen Chemie [Methods of Organic Chemistry], Georg Thieme Verlag, Stuttgart or (ii) Comprehensive Organic Synthesis, Ed. B. M. Trost, Pergamon Press, Oxford, 1991. The carboxylic acid is preferably activated as mixed anhydride, such as, for example, AG=iso-butyl-carbonate; as N-carboxyanhydride (R⁵ and AG=—CO—); or by a coupling agents such as, for example dicyclohexylcarbodiimid (DCC), 1-ethyl-3-(3′-dimethylaminopropyl)carbodiimide×HCl (EDCI), 2-(7-aza-3-oxido-1H-1,2,3-benzotriazol-1-yl)-1,1,3,3-tetramethyluronium hexafluorophosphate. Other activated carboxylic acid derivatives such as, for example symmetric anhydrides, halides, or activated esters e.g. succinyl or pentafluorophenyl esters may also be employed.

In the above scheme PG¹ stands for a suitable protecting group of the amino group that is stable under the respective reaction conditions. Protecting groups of this type are known to the person skilled in the art and are described in detail in T. W. Greene, P. G. Wuts, Protective Groups in Organic Synthesis, 3^{rd} ed., John Wiley, New York, 1999. The amino group is preferably protected by carbamates, PG¹ being for example tert-butyloxycarbonyl (Boc), 9-fluorenylmethyloxycarbonyl (FMOC) or benzyloxy-carbonyl (Cbz-/Z-) or other oxycarbonyl derivatives.

In the above scheme PG² stands for a suitable protecting group of the carboxyl group or COOPG² stands for the carboxylic group attached to a polymeric resin suitable for solid phase synthesis. Protecting groups of this type are known to the person skilled in the art and are described in detail in T. W. Greene, P. G. Wuts, Protective Groups in Organic Synthesis, 3^{rd} ed., John Wiley, New York, 1999. The carboxyl group is preferably esterified, PG² being C₁₋₆-alkyl such as, for example, methyl, ethyl, propyl, isopropyl, butyl, isobutyl, t-butyl, pentyl, isopentyl, neopentyl, hexyl, a C₃₋₇-cycloalkyl such as, for example, cyclopropyl, cyclopropylmethyl, cyclobutyl, cyclopentyl, cyclohexyl, an aryl such as, for example, phenyl, benzyl, tolyl or a substituted derivative thereof.

Step A

Formation of the amides (IV) can take place by reacting an activated form of the respective carboxylic acid (II), such as a N-carboxyanhydride or an iso-butylcarbonate with the desired amine (III) or an acceptable salt thereof.

N-carboxyanhydrides of (II) are commercially available or can be prepared for example by the reaction of the Bis-(N-tert-butyloxycarbonyl) protected derivative of (II) with thionylchloride and pyridine in dimethylformamide or by the reaction of the free amino acid of (II) with phosgene or with phosgene equivalents such as diphosgene, triphosgene or methylchloroformate. Iso-butylcarbonates can be prepared in situ by reaction of the N-protected amino acid (II) with iso-butylchloroformate as described below. Activated derivatives of the acids (II) such as other anhydrides, halides, esters e.g. succinyl or pentafluorophenyl esters or activated carboxylic acids obtained by the reaction with coupling agents such as, for example dicyclohexylcarbodiimid (DCC), 1-ethyl-3-(3′-dimethylaminopropyl)carbodiimide×HCl (EDCI), 2-(7-aza-3-oxido-1H-1,2,3-benzotriazol-1-yl)-1,1,3,3-tetramethyluronium hexafluorophosphate may also be employed.

For example, amides of type (IV) can be prepared as follows:

1) N-carboxyanhydride Procedure

A solution/suspension of the amine (III), the N-carboxyanhydride of (II) and catalytic amounts of 4-(N,N′-dimethylamino)pyridine in an inert solvent was refluxed for 0.5-14 days with exclusion of moisture. The product was either isolated by filtration or by aqueous workup employing standard procedures. If necessary the product was purified by trituration or by flash-chromatography or used without further purification.

2) Mixed Anhydride Procedure

A solution of the carboxylic acid derivative (II) and of N-methylmorpholine in an inert solvent was cooled to −15° C. and iso-butyl chloroformate was added and stirred at 0° C. The amine (III) in an inert solvent was added at −15° C. The solution was stirred at 0° C., and at r.t. and was evaporated. The residue was redissolved in ethyl acetate, washed with aqueous acid and base, dried and evaporated. If necessary the product was purified by trituration or by flash-chromatography or used without further purification.

Compounds of general formula (II) are commercially available, known or can be prepared by customary methods starting from known α-amino acids or precursors for customary α-amino acid synthesis. For the preparation process according to the invention, the amino group is in this case blocked by a conventional protective group PG¹.

In the α-position to the carboxyl group, these carboxylic acid derivatives can have substituents such as described under R³ and R⁴, for example, hydrogen, a C₁-C₁₀-alkyl, a C₃-C₇-cycloalkyl, an aryl, an alkenyl residue, or an alkinyl residue. The alkyl, alkenyl and cycloalkyl residues and the benzyl residue can be introduced by reaction of the ester of the starting compounds with the appropriate alkyl, alkenyl, cycloalkyl or benzyl halides in basic medium, if the corresponding derivatives are not commercially available. The alkinyl residue can be introduced, for example, by reaction of the bromo ester of the present starting compound with an appropriate acetylide anion. In the case of the phenyl residue the starting materials used are preferably the corresponding α-phenyl-α-aminocarboxylic acid derivatives and, if necessary, the other substituents at the α-C atom to the terminal carboxyl group are introduced via the appropriate alkyl halide.

The above reactions and their implementation are well known to the person skilled in the art and are described in detail in standard textbooks such as, for example, in (i) Houben-Weyl, Methoden der organischen Chemie [Methods of Organic Chemistry], Georg Thieme Verlag, Stuttgart or Stuttgart or (ii) Comprehensive Organic Synthesis, Ed. B. M. Trost, Pergamon Press, Oxford, 1991.

If the substituents themselves should be substituted, e.g. by R′, appropriate reactive groups should be present in the substituent to allow further functionalization. These reactive groups should be inert to the reaction conditions of the previous step. For this purpose, the substituent can also be unsaturated to allow further functionalization such as palladium catalyzed C—C-coupling reactions (e.g. Heck-reaction or Sonoga-shira-reaction), eventually followed by hydrogenation (scheme 2):

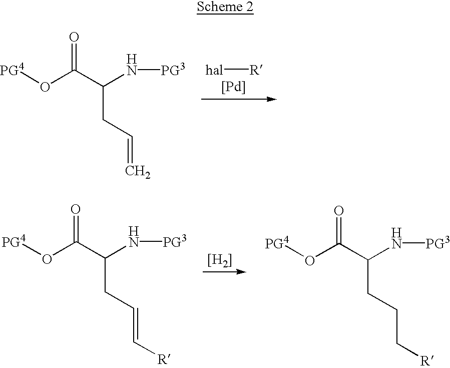

In the abovementioned scheme PG⁴ stands for a protecting group of the carboxyl group as described under PG², hal stands for a leaving group such as a halogen, tosyl, mesyl or triflate, [Pd] stands for a Palladium(0) or Palladium(II) moiety. PG³ stands for a protecting group of the amino group such as described under PG¹. Protecting groups of this type are known to the person skilled in the art and are described in detail in T. W. Greene, P. G. Wuts, Protective Groups in Organic Synthesis, 3^{rd} ed., John Wiley, New York, 1999.

If the substituent R³ or R⁴ in the α-position to the carboxylic group carry an appropriate substituted aryl or heteroaryl unit, another method for insertion of an additional substituent are the C—C-coupling reactions as described under the synthesis of precursors (VI).

Compounds of general formula (III) are commercially available, known or can be prepared by customary methods starting from known carboxylic acid derivatives.

In case R¹⁻¹, R¹⁻² and/or R¹⁻³ are methylen groups, the carbon chain can be elongated by Arndt-Eistert-reaction and optionally be derivatized by common methods for α-derivatization of carboxylic acids such as nucleophilic substitution.

In case, Y is different from carbonyl and/or D is different from nitrogen—as defined in formula (I)—the respective compounds (IV) can be prepared as follows:

For example, in case Y and D form an sulfinamide, or sulfonamide, they may be prepared by reacting the respective sulfinylchlorides or sulfonylchlorides with the desired amine (III) or an acceptable salt thereof.

For example, in case Y and D form an ether or thioether, the O—C or S—C— bonds are formed via alkylation of the corresponding alcohols or thiols with alkylating agents such as alkyl halides, alkyl tosylates and the like. The thioether can be converted into the corresponding sulfoxides or sulfones by oxidation with reagents like mCPBA or hydrogen peroxide.

In case Y and D form a carbon-nitrogen-bond or a nitrogen-carbon-bond, the bond is established by reductive amination via the corresponding aldehyde or ketone and the corresponding amine in the presence of a reducing agent such as sodium cyanoborohydride. In the case Y and D form a carbon-nitrogen bond in which the nitrogen atom is attached to an aromatic ring, the amine group —Y—NR²H can be coupled to the aromatic ring by an Buchwald reaction employing an halogen or triflate substituted aromatic residue and a suitable catalyst such as, for example Pd(0) or Pd(II) with phospine ligands such as triphenylphosphine, 2,2′-bisdiphenylphosphino)-1,1′-bi-naphthyle (BINAP) or 1,1′-bis-(diphenylphosphino)ferrocene (dppf) together with an appropriate base such as, for example cesium carbonate or cesium fluoride.

In case Y and D form a carbon-carbon-bond, the bond may be established by Wittig reaction of the corresponding ketone or aldehyde and the corresponding phosphonium ylide followed by reduction of the double bond, e.g. by catalytic hydrogenation.

In case Y is carbonyl and D is a carbon moiety, the bond may be formed by a Grignard type reaction of the corresponding aldehyde of Y and the corresponding Grignard-reagent of D, followed by the oxidation of the resulting alcohol to the ketone, e.g. by Swern-oxidation or Jones-oxidation.

The above reactions and their implementation are well known to the person skilled in the art and are described in detail in standard textbooks such as, for example, in (i) Houben-Weyl, Methoden der organischen Chemie [Methods of Organic Chemistry], Georg Thieme Verlag, Stuttgart Stuttgart or (ii) Comprehensive Organic Synthesis, Ed. B. M. Trost, Pergamon Press, Oxford, 1991.

When more than one choice of reaction methods exist, the person skilled in the art is able to choose the appropriate pathway according to selectivity and possible use of protecting groups such as described in T. W. Greene, P. G. Wuts, Protective Groups in Organic Synthesis, 3^{rd} ed., John Wiley, New York, 1999.

Step B

The removal of protecting group PG¹ can be performed, depending on the nature of PG¹, either by an acid such as trifluoroacetic acid (for example in the case PG¹ is tert-butyloxycarbonyl (Boc)), a base such as piperidine (for example in the case PG¹ is 9-fluorenylmethyloxycarbonyl (FMOC)) or by catalytic hydrogenation (for example in the case PG¹ is benzyloxycarbonyl (Cbz-/Z-)).

Step C

Formation of the amides (VII) can take place by reacting the respective carboxylic acids (VI)—activated by a coupling agent such as DCC and HOBt; EDCI and HOBt or HATU—with the desired amines (V) or an acceptable salt thereof. Activated derivatives of the acids (VI) such as anhydrides, halides, and esters e.g. succinyl or pentafluorophenyl esters may also be employed.

For example, amides (VII) can be prepared as follows:

A solution of carboxylic acid, HOBt and EDCI in an inert solvent is stirred at r.t. After addition of the amine and a non-nucleophilic base such as ethylisopropylamine stirring is continued at r.t. or elevated temperature. The reaction mixture is poured into water and worked up by standard procedures.

Compounds of general formula (VI) are commercially available, known or can be prepared by customary methods starting from known carboxylic acid derivatives.

For example, biphenyl substituted acetic acid derivatives can be prepared by means of an aryl-aryl coupling of the respective phenyl acetic acid derivatives and a suitable phenyl system.

Possible coupling reactions are, for example, the reaction of two unsubstituted phenyl groups in the presence of AlCl₃ and an acid (Scholl reaction), the coupling of the two phenyl iodides in the presence of copper (Ullmann reaction), the reaction of the unsubstituted carboxylic acid derivative with a phenyldiazonium compound under basic conditions (Gomberg-Bachmann reaction) or coupling with participation of organometallic reagents such as coupling of a phenyl halide with an organometallic phenyl compound in the presence of a palladium compound, for example a Pd(0), a Pd(II) or a Pd(IV) compound, and of a phosphane such as triphenylphosphane (e.g. Suzuki reaction).

Bisarylureas can be prepared by coupling of an amino phenyl acetic acid derivative and a phenylisocyanate. Bisarylamides can be prepared by coupling of an amino phenyl acetic acid and an activated benzoic acid derivative such as described under Step A. Bisarylcarbamates can be prepared by coupling of an isocyanato phenyl acetic acid ester and a phenol derivative followed by saponification as described in Step D.

In case, A—as defined in formula (I)—is different from carbonyl, the respective compounds (IV) can be prepared as follows:

For example, in case A forms a sulfinamide, sulfonamide, they may be prepared as described under Step A. Oxalic amides can be prepared by the same means as the amides described above. Phosphinic acid amides and phosphonic acid amides can be prepared by coupling of activated phosphinic/phosphonic acids with amines (V). In case A is a heteroaromatic or aromatic system, the respective compounds (IV) can be prepared by nucleophilic substitution of the respective fluorosubstituted systems with a suitable amine (V).

Step D

The removal of the protecting group PG² can be performed either by an acid such as trifluoroacetic acid or an base such as potassium hydroxide or lithium hydroxide, depending on the nature of PG². Reactions are carried out in aqueous, inert organic solvents such as alcohols e.g. methanol or ethanol, ethers e.g. tetrahydrofurane or dioxane or polar aprotic solvents e.g. dimethylformamide. If necessary, mixtures of the above solvents may be used.

In case PG² stands for polymeric resin, the removal can take place using strong acid such as trifluoroacetic acid in dichloromethane.

### EXAMPLES

Abbreviations

| | |
|---|---|
| AcOH | acetic acid |
| Boc | tert-butyloxycarbonyl |
| DCC | dicyclohexylcarbodiimid |
| GC | gas chromatography |
| DIPEA | diisopropylethylamine |
| EDGI | 1-ethyl-3-(3'-dimethylaminopropyl)carbodiimidexHCl |
| eq. | equivalents |
| FC | flash chromatography |
| HATU | 2-(7-aza-3-oxido-1H-1,2,3-benzotriazol-1-yl)-1,1,3,3- |
| | tetramethyluro-nium hexafluorophosphate |
| HOBt | N-hydroxybenzotriazole monohydrate |
| HPLC | high performance liquid chromatography |
| ICAM-1 | intracellular adhesion molecule 1 |
| IL-1 | interleukin 1 |
| LPS | lipopolysaccharide |
| MAdCAM-1 | mucosal addressin cell adhesion molecule 1 |
| MeOH | methanol |
| min. | minutes |
| M.p. | melting point |
| NF-kB | nuclear factor kB |
| NMR | nuclear magnetic resonance |
| n.d. | not determined |
| r.t. | room temperature |
| R_{f} | TLC: R_{f} value = distance spot traveled/distance solvent |
| | front traveled |
| TFA | trifluoroacetic acid |
| THF | tetrahydrofurane |
| TLC | thin layer chromatography |
| TINF-α | tumor necrosis factor α |
| t_{R} | retention time determined by HPLC |
| VCAM-1 | vascular cell adhesion molecule 1 |
| VLA-4 | very late antigen 4 (α₄β₁ integrin) |

General Remarks

In the examples below, all quantitative data, if not stated otherwise, relate to percentages by weight.

Flash chromatography was carried out on silica gel 60, 40-63 μm (E. Merck, Darmstadt, Germany).

Thin layer chromatography was carried out, employing silica gel 60 F₂₅₄ coated aluminum sheets (E. Merck Darmstadt, Germany) with the mobile phase indicated.

Melting points were determined in open capillaries and are not corrected.

All retention times are indicated in minutes and, if not stated otherwise, were determined by high-performance liquid chromatography (HPLC) by means of UV detection at 210/250 nm and a flow rate of 1 ml/min. An acetonitrile/water mixture with 0.1% trifluoroacetic acid (vol./vol.) was used as eluent with a linear gradient of: 0 min.=0% acetonitrile, 25 min.=100% acetonitrile, 31 min=100% acetonitrile, 32 min 0% acetonitrile, 38 min 0% acetonitrile. Two methods were used: for method A a LiChrospher 100 RP-18, 5 μm, 250×4 mm (E. Merck, Darmstadt, Germany) column and for method B a Purospher RP-18e, 5 μm, 250×4 mm (E. Merck, Darmstadt, Germany) column was used.

The mass determinations were carried out using the electron spray ionization (ESI) method employing loop injection or split injection via a HPLC system.

Precursor Synthesis

### Example I

### 2-{4-[(2-Toluidinocarbonyl)amino]phenyl}acetic Acid

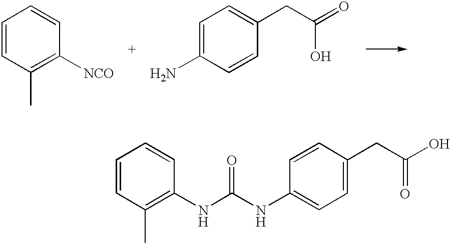

To a solution of 2-(4-aminophenyl)acetic acid (108.8 g, 0.72 mol) in CH₂Cl₂ (1.0 l) and triethylamine (120 ml) was added a solution of 2-methylphenyl isocyanate (90.5 ml, 0.72 mol) in CH₂Cl₂ (500 ml) dropwise at r.t. After stirring for 18 h at r.t., water (2.5 l) and CH₂Cl₂ (2.0 l) were added and the layers were separated. The organic layer was extracted with water (3×400 ml). The combined aqueous layers were concentrated to 3.0 l and acidified to pH 2 by the addition of concentrated aqueous HCl. The precipitate was collected by filtration, washed with cold water and dried in an exsiccator over concentrated H₂SO₄ affording 166.5 g (82%) white solid. M.p. 205-206° C.; TLC (CH₂Cl₂/MeOH 9:1): R_{f} 0.14. ¹H-NMR (400 MHz, D₆-DMSO): 12.21 (br s, 1H), 9.11 (s, 1H), 8.00 (s, 1), 7.83 (d, 7.6 Hz, 11, 7.40 (d, 8.5 Hz, 2H), 7.17-7.12 (m, 4), 6.96-6.92 (m, 111, 3.48 (s, 2H), 2.24 (s, 3H).

### Example II

### 2-{4-1-(2-Toluidinocarbonyl)amino]phenyl}acetyl-L-leucine Methyl Ester

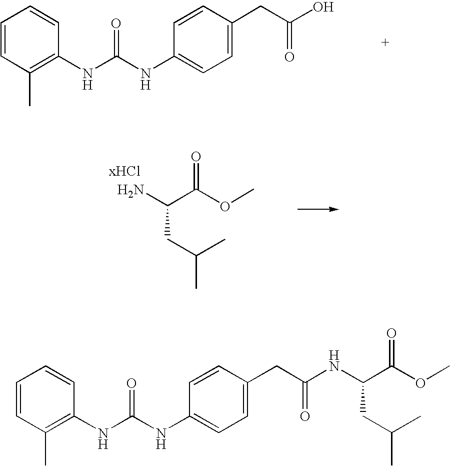

A solution of 2-{4-[(2-toluidinocarbonyl)amino]phenyl}acetic acid (1.96 g, 6.89 mmol), HOBt (1.16 g, 7.58 mmol) and EDCI in 70 ml dimethylformamide was stirred 90 min at r.t. After addition of L-leucine methyl ester hydrochloride (1.25 g, 6.89 mmol) in dimethylformamide (20 ml) and ethyldiisopropylamine (5.75 ml, 34.5 mmol) stirring at r.t. was continued for 18 h. The reaction mixture was poured into water (350 ml) and extracted with ethyl acetate (4×150 ml). The combined organic layers were washed with 0.1 N aqueous HCl, saturated aqueous Na₂CO₃, brine, dried (MgSO₄) and evaporated. Yield. 2.49 g (88%) white solid. M.p. 166-168° C.; TLC (CH₂Cl₂/MeOH 9:1): R_{f} 0.56; ¹H-NMR (400 MHz, D₆-DMSO): 8.96 (s, 1H), 8.42 (d, 7.7 Hz, 1H), 7.89 (s, 1H), 7.84 (d, 7.44 Hz, 1H), 7.38 (d, 8.5 Hz, 2H), 7.18-7.11 (m, 4H), 6.96 (m, 1H), 4.30-4.23 (m, 1H), 3.61 (m, 3H), 3.43-3.36 (m, 2H), 2.24 (s, 3H), 1.67-1.45 (m, 3H), 0.89 (d, 6.4 Hz, 3H), 0.82 (d, 6.4 Hz, 3H).

### Example III

### 2-{4-[(2-toluidinocarbonyl)amino]phenyl}acetyl-L-leucine

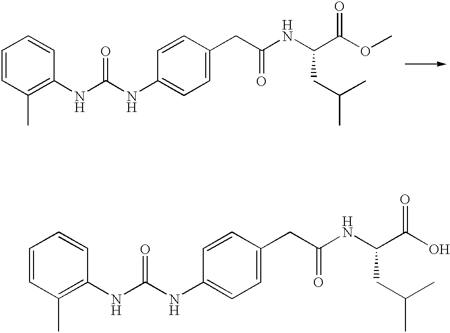

A solution of 2-{4-[(2-toluidinocarbonyl)amino]phenyl}acetyl-L-leucine methyl ester (2.42 g, 5.88 mmol) and KOH (3.30 g, 58.75 mmol) in methanol/water 1:1 (180 ml) was stirred at 50° C. for 5 h. After washing with methyl-tert-butylether (80 ml) the volume of the reaction mixture was reduced until a slight turbidity was observed. The solution was acidified to pH 2 by the addition of 1 N aqueous HCl. The precipitate was collected by filtration, washed with cold water and dried in vacuum. Yield: 1.75 g (72%) white solid. M.p.: 178-179° C., TLC (CH₂Cl₂/MeOH/AcOH 9:1:0.1): R_{f} 0.16; ¹H-NMR (400 MHz, D₆-DMSO): 12.51 (br s, 1H), 9.00 (s, 1H), 8.25 (d, 8.0 Hz, 1H), 7.93 (s, 1H), 7.83 (d, 7.5 Hz, 1H), 7.36 (d, 8.5 Hz, 2H), 7.17-7.12 (m, 4H), 6.95-6.91 (m, 1H), 4.234.17 (m, 1H), 3.43-3.32 (m, 2H), 2.24 (s, 3H), 1.68-1.46 (m, 3H), 0.89 (d, 6.5 Hz, 3H), 0.82 (d, 6.5 Hz, 3H).

### Example IV

### Methyl 4-({[(3-methoxypropyl)amino]acetyl}amino)benzoate

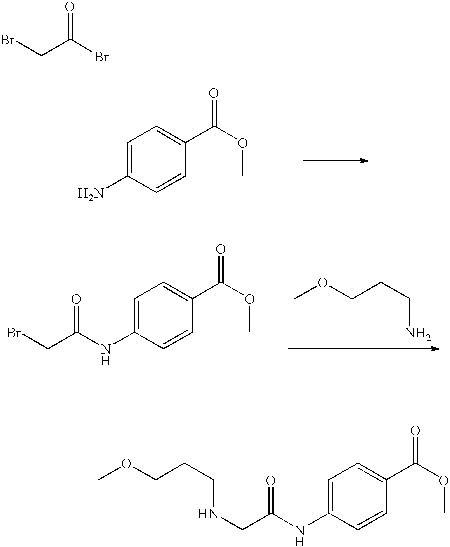

To a solution of methyl 4-aminobenzoate (10.0 g, 66.2 mmol) and triethylamine (10.1 ml, 72.8 mmol) in dichloromethane (100 ml) was added a solution of bromo-acetylbromide (6.34 ml, 72.8 mmol) in dichloromethane (30 ml) at 0° C. After stirring for 18 h at room temperature and 18 h under reflux the reaction mixture was concentrated under vacuum. The residue was taken up in ethyl acetate, washed with 1 N aqueous HCl and water, dried over MgSO₄ and evaporated. Yield 15.8 g (88%) of methyl 4-[(bromoacetyl)amino]benzoate as a pale brown solid. M.p.: 144-146° C., TLC (hexane/ethyl acetate 1:1): R_{f} 0.46.

To a solution of methyl 4-[(bromoacetyl)amino]benzoate (2.72 g, 10.0 mmol) in dimethylformamide (20 ml) was added 3-methoxypropylamine (1.78 g, 20.0 mmol) and triethylamine (22.3 ml, 160 mmol). After stirring at room temperature for 18 h, the reaction mixture was concentrated under vacuum and purified by flash chromatography (CH₂Cl₂/MeOH 9:0.4) affording 1.81 g (65%) of methyl 4-({[(3-methoxypropyl)amino]acetyl}amino)benzoate as a pale red solid.

### Example V

### 4-(1H-tetraazol-5-yl)aniline

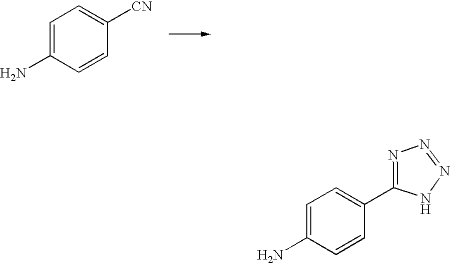

To a solution of 4-aminobenzonitrile (11.8 g, 100 mmol) and triethylamine hydrochloride (17.9 g, 130 mmol) in toluene (550 ml) was added sodium azide (8.45 g, 130 mmol). After stirring for 24 h at 95° C., the reaction mixture was cooled to room temperature and was extraced with water (3×60 ml). The combined aqueous phases were acidified with concentrated aqueous HCl to pH 2-3. The product was collected by filtration, washed with water and dried in vacuum. Yield 9.59 g (60%) pale brown solid. M.p.: 280-281° C., TLC (CH₂Cl₂/MeOH/AcOH 9:1:0.1): R_{f} 0.30

### Example VI

### Ethyl 1,2,3,4-tetrahydro-6-quinolinecarboxylate

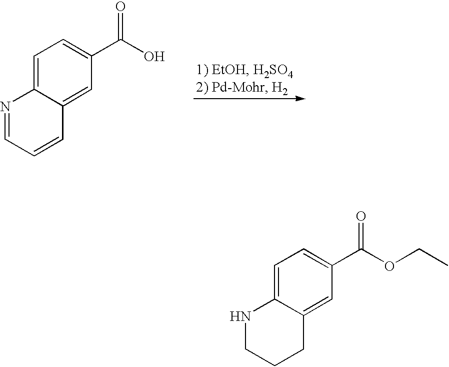

A solution of 6-quinolinecarboxylic acid (9.50 g, 54.9 mmol) and 2 ml of concentrated sulfuric acid in ethanol (250 ml) was refluxed for 8 h. The solvent was evaporated and the residue was taken up in water. After adjustment of the pH to 8 by the addition of potassium hydroxide the product was collected by filtration and dried in vacuum. Yield 9.85 g (89%) of ethyl 6-quinolinecarboxylate as a pale brown solid. M.p.: 66-67° C., TLC (CH₂Cl₂/MeOH/AcOH 9:0.5:0.1): R_{f} 0.52

A solution of ethyl 6-quinolinecarboxylate (9.80 g, 48.7 mmol) was acidified to pH 2 by the addition of 1N aqueous HCl. After addition of 20% Pd-Mohr catalyst (1.96 g) the solution was hydrogenated at 60° C. under 3 bar of hydrogen pressure for 17 h. The reaction mixture was filtered through celite. The filtrate was evaporated and the residue was taken up in ethyl acetate and water. The pH was adjusted to 16 by the additon of 1 N aqueous potassium hydroxide. The phases were separated and the organic phase was washed with brine, dried over Na₂SO₄ and evaporated. Yield 8.72 g (87%) of ethyl 1,2,3,4-tetrahydro-6-quinolinecarboxylate as a pale brown solid. M.p.: 68-70° C., GC-MS: [M⁺]=205.

Compound Synthesis

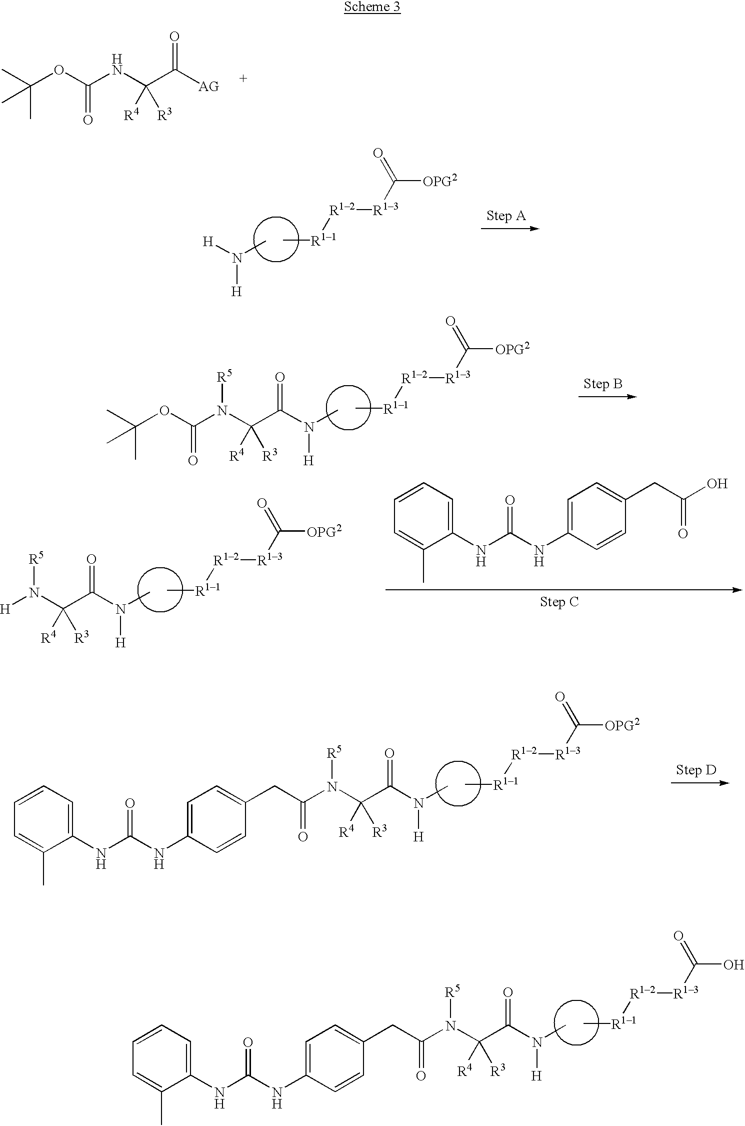

Step A:

General Procedure A1 (GP A1): Coupling of Amines with Boc-L-leucin-N-carboxyanhydride:

A solution/suspension of 1.0 eq. of the amine, 1.0 eq. of Boc-L-leucin-N-carboxyanhydride and 0.3 eq. of 4-(N,N′-dimethylamino)pyridine was refluxed for 0.5-14 days with exclusion of moisture. If a precipitate was formed, the precipitate (product) was collected by filtration. The reaction mixture/filtrate was evaporated to dryness, redissolved in ethyl acetate and washed with 1 N aqueous HCl, saturated aqueous NaHCO₃ and brine, dried over MgSO₄ and evaporated. Both solids were combined.

If necessary the product was purified by trituration or by flash-chromatography or used without further purification.

### Example 1

### Methyl 4-({Boc-L-leucine}amino)benzoate

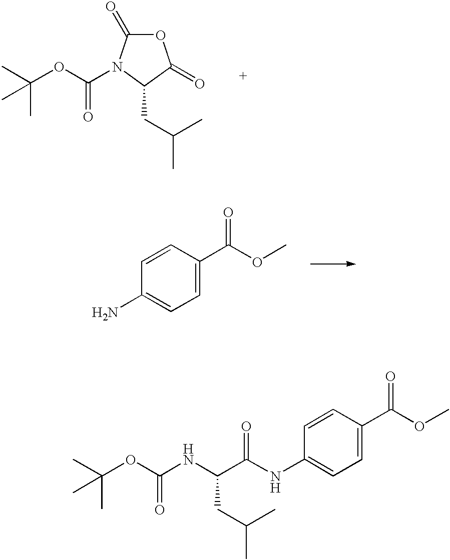

Methyl 4-aminobenzoate (0.75 g, 4.97 mmol) was dissolved in CH₂Cl₂ (7 ml). After the addition of Boc-L-leucin-N-carboxyanhydride (1.28 g, 4.79 mmol) and 4-(N,N′-dimethylamino)pyridine (180 mg, 1.49 mmol) the solution was stirred under reflux for 4 days. The precipitate (product) was collected by filtration. The filtrate was evaporated to dryness, redissolved in ethyl acetate and washed with 1 N aqueous HCl, saturated aqueous NaHCO₃ and brine, dried over MgSO₄ and evaporated. Combined Yield: 1.35 (75%) white solid.

General Procedure A2 (GP A2): Coupling of Amines with Carboxylic Acids Activated by Iso-Butyl Chloroformate.

A solution of 1.0 eq. of the carboxylic acid derivative and 1.0 eq. of N-methylmorpholine in tetrahydrofurane was cooled to −15° C. and 1.0 eq. of iso-butyl chloroformate was added dropwise. After 5 min at 0° C., 1.0 eq. of the amine in tetrahydrofurane was added at −15° C. The solution was stirred for 1 h at 0° C., 1-4 d at r.t. and was evaporated. The residue was redissolved in ethyl acetate, washed with 1 N aqueous HCl (2×), saturated aqueous NaHCO₃ and brine, dried over MgSO₄ and evaporated.

TABLE 1
Characterization of reaction products according to Step A

| Example | | | | | | | | HPLC |
|---|---|---|---|---|---|---|---|---|
| No. | Structure | Procedure | Yield [%] | Product | R_{f} | M.p. [° C.] | ESI-MS | t_{R}[min] |
| 1 | 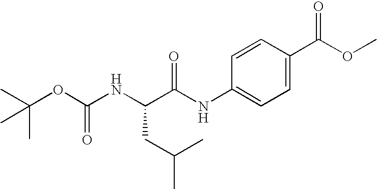 | GP A1 | 75 | white solid | 0.52 (CH₂Cl₂/MeOH 9:0.1) | 72-73 | 309.1 [M + H]⁺ | n.d. |
| 2 | 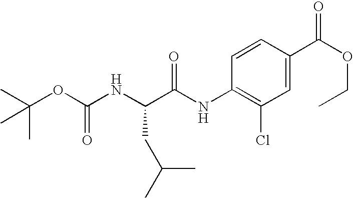 | GP A1, FC: CH₂Cl₂/MeOH 9:0.3-8:2 | 31 | white solid | 0.68 (petrol ether/ethyl acetate 8:2) | n.d. | 413.4 [M + H]⁺ | 27.5 Method B |
| 3 | 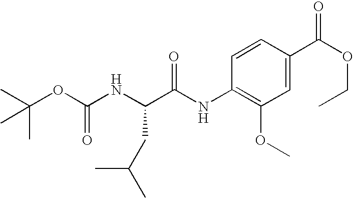 | GP A1, crude product | 42 | pale red solid | 0.60 (petrol ether/ethyl acetate 6:4) | n.d. | 409.1 [M + H]⁺ | 25.4 Method B |
| 4 | 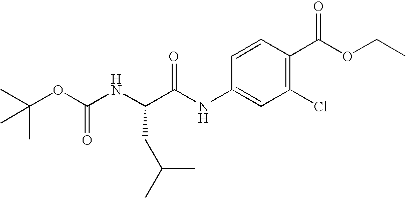 | GP A1, crude product | 39 | pale brown oil | 0.30 (petrol ether/ethyl acetate 8:2) | n.d. | 399.0 [M + H]⁺ | 25.4 Method B |
| 5 | 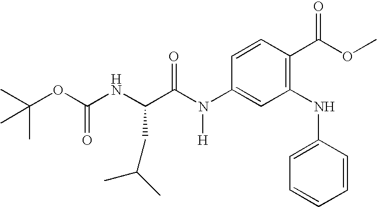 | GP A1, crude product | 52 | white solid | 0.46 (petrol ether/ethyl acetate 8:2) | 207-209 | 456.1 [M + H]⁺ | 27.8 Method B |
| 6 | 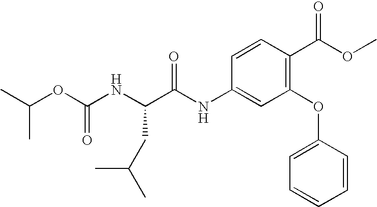 | GP A1, crude product | 52 | white solid | 0.70 (petrol ether/ethyl acetate 8:2) | n.d. | 457.1 [M + H]⁺ | 26.0 Method B |
| 7 | 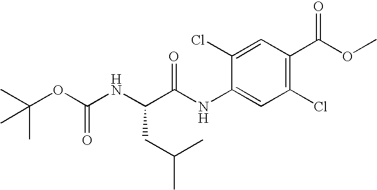 | GP A1, crude product | 37 | pale brown solid | 0.84 (petrol ether/ethyl acetate 6:4) | n.d. | 431.0 [M − H]⁻ | 27.1 Method B |
| 8 | 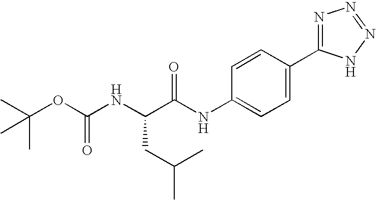 | GP A1 (aq. NaHCO₃ wash omitted) crude product | 39 | pale brown solid | 0.62 (CH₂Cl₂/MeOH/AcOH 9:1:0.1) | n.d. | 373.1 [M − H]⁻ | 21.3 Method A |
| 9 | 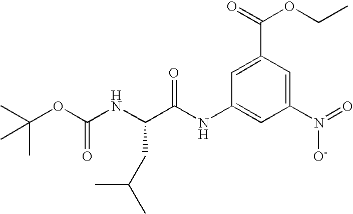 | GP A1, crude product | 46 | pale yellow solid | 0.80 (petrol ether/ethyl acetate 1:1) | n.d. | 424.1 [M + H]⁺ | 25.9 Method B |
| 10 | 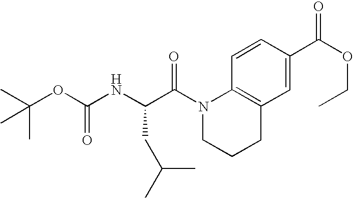 | GP A1, FC: petrol ether/ethyl acetate 9:0.3-9:1 | 3 | yellow oil | 0.52 (petrol ether/ethyl acetate 1:1) | n.d. | 419 [M + H]⁺ | n.d. |
| 11 | 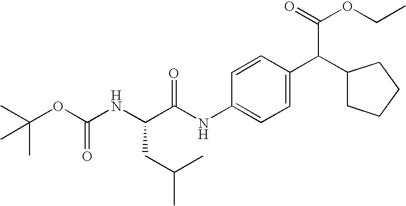 | GP A2, FC: petrol ether/ethyl acetate 9:1 | 17 | pale yellow solid | 0.90 (petrol ether/ethyl acetate 6:4) | n.d. | 461.5 [M + H]⁺ | n.d. |
| 12 | 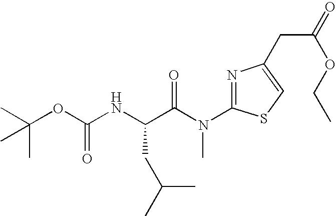 | GP A2, crude product | 89 | pale yellow oil | 0.83 (CH₂Cl₂/MeOH/AcOH 9:1:0.1) | n.d. | 414.3 [M + H]⁺ | 25.3 Method B |
| 13 | 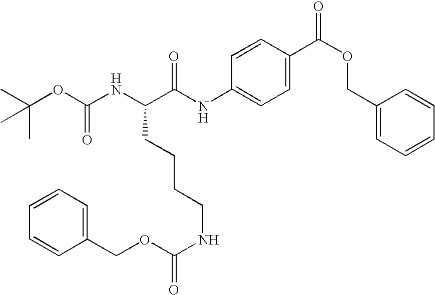 | GP A2, FC: petrol ether/ethyl acetate 10:1-6:4 | 35 | white solid | 0.36 (petrol ether/ethyl acetate 6:4) | 50-52 | 590.4 [M + H]⁺ | n.d. |
| 14 | 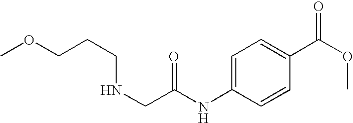 | as described in the precursor synthesis | 65% | pale red solid | 0.36 (CH₂Cl₂/MeOH 9:1) | 49-50 | 281.0 [M + H]⁺ | n.d. |

Step B:

General Procedure B (GP B): Cleavage of the Boc-Protecting Group with Trifluoroacetic Acid

To a solution of the Boc-protected amine was added 20 vol % trifluoroacetic acid in dichloromethane at 0° C. Stirring was continued at room temperature for 0.5-24 h. The solvent was removed at room temperature under reduced pressure. The residue was coevaporated twice with dichloromethane, dried under high vacuum and subjected to the reaction step C without further purification.

Step C:

General Procedure (GP C1): Coupling of Amines with 2-{4-[(2-toluidinocarbonyl)-amino]phenyl}acetic Acid:

A solution of 1.0 eq. 2-{4-[(2-toluidinocarbonyl)amino]phenyl}acetic acid, 1.1 eq. HOBt and 1.1 eq. EDCI in DMF was stirred for 2 h at r.t. After addition of 1.0 eq. amine e.g. as TFA salt and 3-9 eq. ethylisopropylamine stirring was continued for 18 h at r.t. The reaction mixture was poured into the 4-fold amount of water. The precipitate was collected by filtration, washed with cold water and dried in vacuum. If necessary the product was purified by trituration or by flash-chromatography.

Methyl 4-([({4-[(2-toluidinocarbonyl)amino]phenyl}acetyl)L-leucin]amino)benzoate

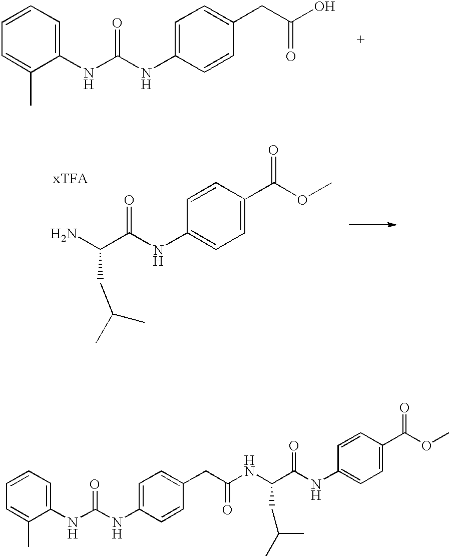

Methyl 4-[(L-leucin)amino]benzoate trifluoroacetate (3.81 g, 10.1 mmol) was reacted according to GP C1 in a total volume of 60 ml of dimethylacetamide. Trituration with CH₂Cl₂ yielded 4.78 g (90%) pale brown solid. M.p. 250-252° C., TLC (AcOH:MeOH:CH₂Cl₂ 0.1:0.5:9): R_{f} 0.46; ¹H-NMR (400 MHz, D₆-DMSO):10.47 (s, 1H), 8.96 (s, 1H), 8.39 (d, 7.7 Hz, 1f), 7.93-7.89 (m, 31), 7.83 (d, 7.8 Hz, 1H), 7.75 (d, 8.8 Hz, 2H), 7.37 (d, 8.4 Hz, 2H), 7.18-7.12 (m, 4H), 6.95-6.92 (m, 1H), 4.49-4.43 (m, 1M), 3.82 (s, 3H), 3.47-3.38 (m, 2H), 2.24 (s, 3H), 1.66-1.50 (m, 3H), 0.92 (d, 6.4 Hz, 3H), 0.86 (d, 6.4 Hz, 3H); ESI-MS: 531.3 [M+H]⁺.

General Procedure (GP C2): Coupling of Amines with 2-{4-[(2-toluidinocarbonyl)amino]phenyl}acetyl-L-leucine

In several cases it is advisable to couple the amine (III) directly with with 2-{4-[(2-toluidinocarbonyl)amino]phenyl}acetyl-L-leucine followed by the cleavage of the protecting group PG², thus omitting steps A and B:

A solution of 1.0 eq. 2-{4-[(2-toluidinocarbonyl)amino]phenyl}acetyl-L-leucine, 1.1 eq. HOBt and 1.1 eq. EDCI in DMF was stirred for 2 h at r.t. After addition of 1.0 eq. amine (as free amino or as a salt) and 3-9 eq. ethylisopropylamine stirring was continued for 18 h at r.t. The reaction mixture was poured into the 4-fold amount of water. The precipitate was collected by filtration, washed with cold water and dried in vacuum. If necessary the product was purified by trituration or by flash-chromatography.

TABLE 2
The following examples were prepared by subsequently applying the general procedures B & C1/C2 as indicated.

| Example | | | Yield | | | M.p. | | HPLC |
|---|---|---|---|---|---|---|---|---|
| No. | Structure | Procedure | [%] | Product | R_{f} | [° C.] | ESI-MS | t_{R}[min] |
| 15 | 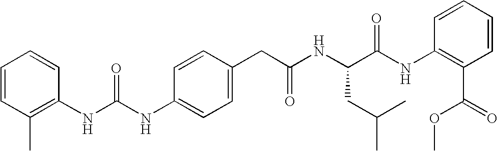 | GP C2 | — | — | — | 170 | — | n.d. |
| 16 | 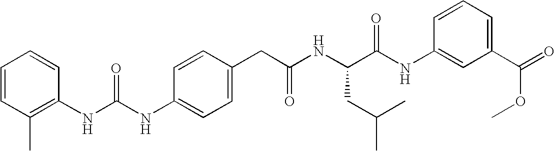 | GP C2 | — | — | — | 220 | — | n.d. |
| 17 | 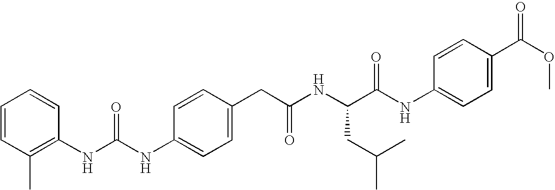 | 1) GP B 2) GP C1, 9 eq. DIPEA | 90 | pale brown solid | 0.46 (CH₂Cl₂/MeOH/AcOH 9:0.5:0.1) | 250-252 | 531.3 [M + H]⁺ | 26.6 Method A |
| 18 | 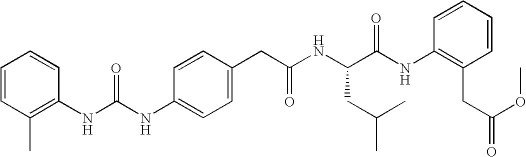 | GP C2 | — | — | — | 218 | — | n.d. |
| 19 | 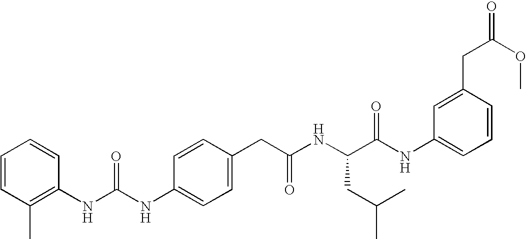 | GP C2 | — | — | — | 200 | — | n.d. |
| 20 | 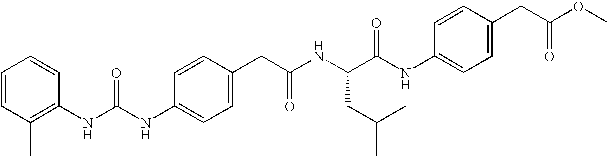 | GP C2 | — | — | — | 222 | — | n.d. |
| 21 | 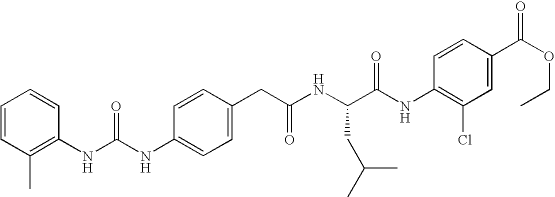 | 1) GP B 2) GP C1, 9 eq. DIPEA | 81 | pale brown solid | 0.74 (CH₂Cl₂/MeOH/AcOH 9:1:0.1) | 163-165 | 579.3 [M + H]⁺ | 25.5 Method B |
| 22 | 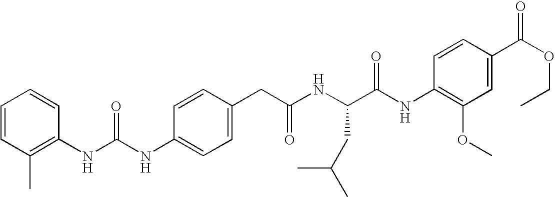 | 1) GP B 2) GP C1, 3) 9 eq. DIPEA | 81 | pale brown solid | 0.34 (CH₂Cl₂/MeOH/AcOH 9:1:0.1) | 139-141 | 575.0 [M + H]⁺ | 24.5 Method A |
| 23 | 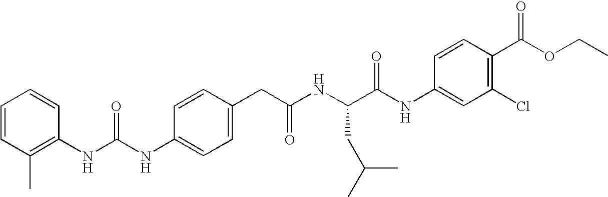 | 1) GP B 2) GP C1: 1.1 eq. HATU (no EDCI, HOBt) & 5 eq. DIPEA | 66 | white solid | 0.74 (CH₂Cl₂/MeOH 9:1) | 181-184 | 564.5 [M + H]⁺ | 24.0 Method A |
| 24 | 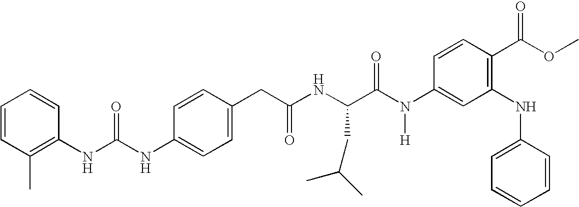 | 1) GP B 2) GP C1, 9 eq. DIPEA | 90 | pale brown solid | 0.34 (CH₂Cl₂/MeOH/AcOH 9:0.5:0.1) | 205-206 | 622.2 [M + H]⁺ | 26.1 Method B |
| 25 | 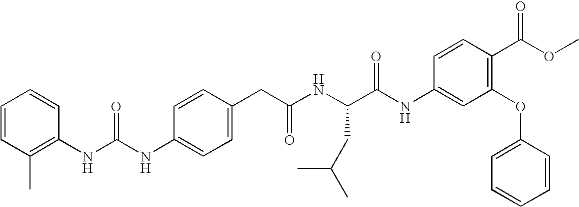 | 1) GP B 2) GP C1, 9 eq. DIPEA | 30 | yellow solid | 0.66 (CH₂Cl₂/MeOH 9:1) | 191-195 | 622.9 [M + H]⁺ | 24.3 Method A |
| 26 | 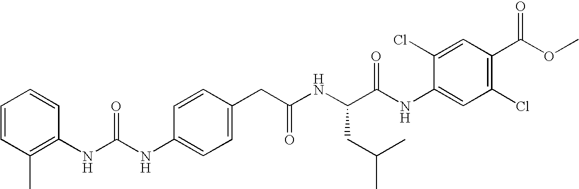 | 1) GP B 2) GP C1, 9 eq. DIPEA | 21 | white solid | 0.34 (petrol ether/ethyl acetate 1:1) | 204-205 | 598.9 [M + H]⁺ | 25.5 Method B |
| 27 | 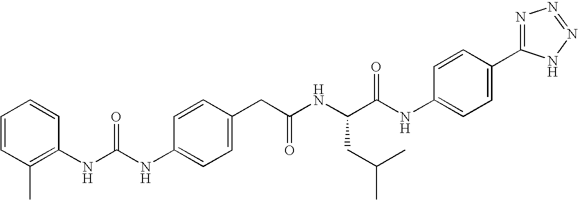 | 1) GP B, 3 eq. Thiohpenole added 2) GP C1, 9 eq. DIPEA | 2 | white solid | 0.16 (CH₂Cl₂/MeOH/AcOH 9.5:0.5:0.1) | 255-257 | 541.2 [M + H]⁺ | 21.4 Method A |
| 28 | 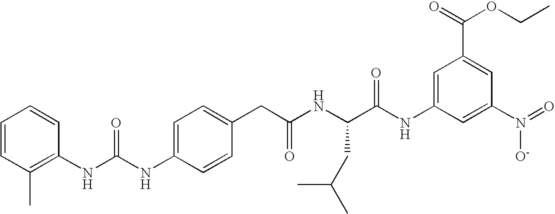 | 1) GP B 2) GP C1, 9 eq. DIPEA | 99 | pale brown solid | 0.80 (CH₂Cl₂/MeOH/AcOH 9:1:0.1) | 90-95 | 590.0 [M + H]⁺ | 25.0 Method B |
| 29 | 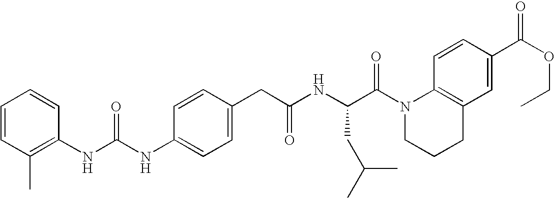 | 1) GP B 2) GP C1, 9 eq. DIPEA | 1.6 | pale yellow oil | 0.68 (CH₂Cl₂/MeOH 9:0.5) | n.d. | 585.2 [M + H]⁺ | n.d. |
| 30 | 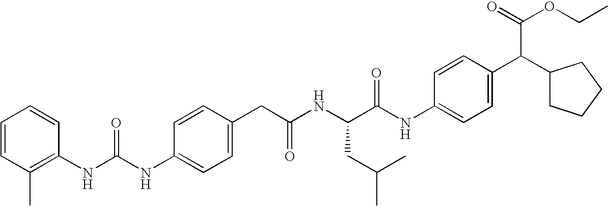 | 1) GP B 2) GP C1, 9 eq. DIPEA | 73 | pale brown solid | 0.66 (CH₂Cl₂/MeOH 9:1) | 186-189 | 627.4 [M + H]⁺ | 26.2 Method A |
| 31 | 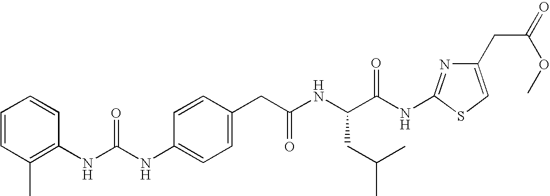 | GP C2 | — | — | — | n.d. | — | n.d. |
| 32 | 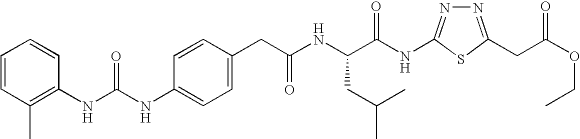 | GP C2 | — | — | — | n.d. | — | n.d. |
| 33 | 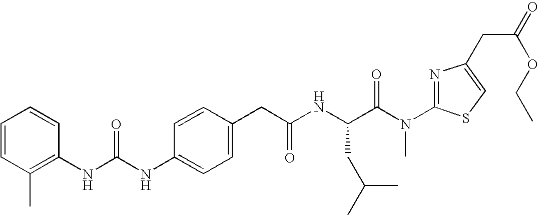 | 1) GP B 2) GP C1, 9 eq. DIPEA | 16 | yellow solid | 0.90 (CH₂Cl₂/MeOH 9:1) | 125-130 | 580.2 [M + H]⁺ | 24.2 Method A |
| 34 | 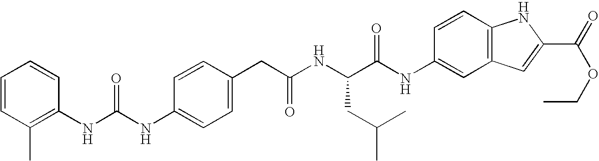 | GP C2 | — | — | — | n.d. | — | n.d. |
| 35 | 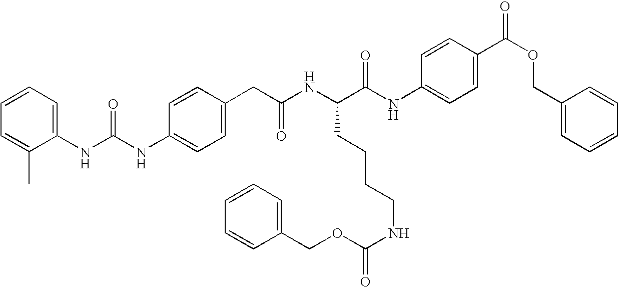 | 1) GP B 2) GP C1, 9 eq. DIPEA | 97 | pale brown solid | 0.62 (CH₂Cl₂/MeOH/AcOH 9:1:0.1) | 188-189 | 756.4 [M + H]⁺ | n.d. |
| 36 | 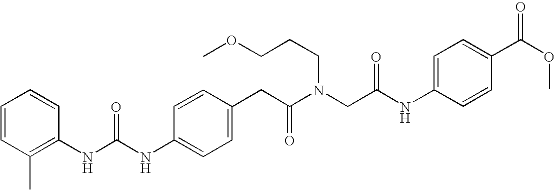 | 1) GP B 2) GP C1, 3 eq. DIPEA | 64 | white solid | 0.50 (CH₂Cl₂/MeOH 9:1) | 197-198 | 547.0 [M + H]⁺ | 21.8 Method A |
| 37 | 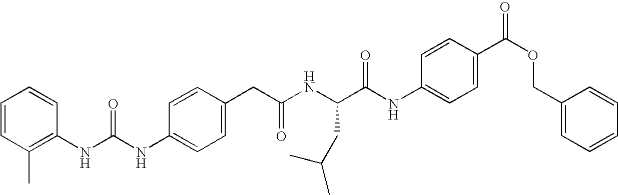 | GP C2, 3 eq. DIPEA | 82 | white solid | 0.80 (CH₂Cl₂/MeOH 9:1) | 188-189 | 613.3 [M + H]⁺ | n.d. |
| 38 | 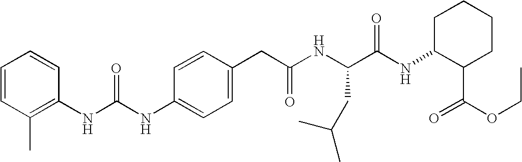 | GP C2 | — | — | — | n.d. | — | n.d. |

Step D

General Procedure D1 (GP D1): Ester Saponification

A solution or suspension of the ester and 1.1 eq. KOH in water/ethanol, methanol and/or dioxane was stirred at 25-50° C. for 2-24 h. After washing with methyl-tert-butylether (80 ml) the volume of the reaction mixture was reduced until a slight turbidity was observed. The solution was acidified to pH 2 by the addition of 1 N aqueous HCl. The precipitate was collected by filtration, washed with cold water and dried in vacuum.

General Procedure D2 (GP D2): Deprotection of Benzyl Esters/Benzyl Carbamates

A solution or suspension of the ester and 10% Pd—C (10%) in dimethylformamide was hydrogenated for 12 h at r.t. and 50 bar hydrogen pressure. The reaction mixture was filtered through celite. Evaporation of the filtrate and purification of the crude product by preparative HPLC (LiChrospher RP-18, 12 μM, 250×25 mm; flow rate 40 ml/min; eluent: acetonitrile/water mixture with 0.1% trifluoroacetic acid (vol./vol.), linear gradient of: 0 min.=40% acetonitrile, 20 min.=80% acetonitrile) afforded the product.

General Procedure D3 (GP D3): Deprotection of Benzyl Esters

A solution or suspension of the ester and 10% Pd—C (10%) in tetrahydrofarane was hydrogenated for 18 h at rt. under atmospheric hydrogen pressure. The reaction mixture was filtered through celite. Evaporation of the filtrate afforded the product.

TABLE 3
following examples were prepared according to the general procedures D1-D3:

| Example | | | Yield | | | | | |
|---|---|---|---|---|---|---|---|---|
| No. | Structure | Procedure | [%] | Product | R_{f} | M.p. [° C.] | ESI-MS | HPLC t_{R}[min] |
| 39 | 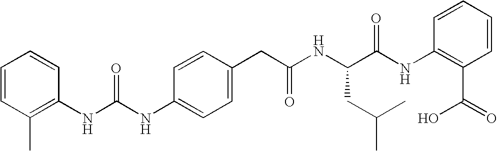 | GP D1; 10 eq. KOH | — | — | — | 190 | — | n.d. |
| 40 | 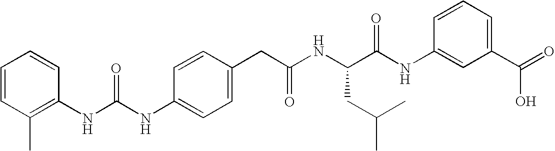 | GP D1; 10 eq. KOH | — | — | — | 220 | — | n.d. |
| 41 | 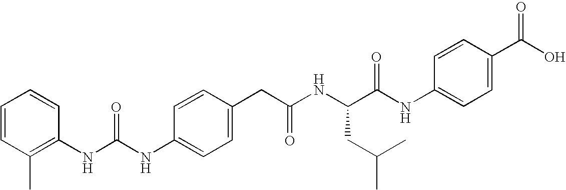 | GP D1; 10 eq. KOH | 90 | white solid | 0.24 (CH₂Cl₂/MeOH/AcOH 9:1:0.1) | 219-223 | 517.0 [M + H]⁺ | 21.3 Method A |
| 42 | 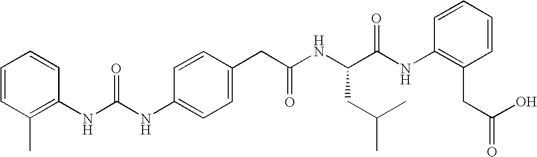 | GP D1; 10 eq. KOH | 87 | yellow solid | 0.06 (CH₂Cl₂/MeOH/AcOH 9:1:0.1)) | 186-190 | 531 [M + H]⁺ | 26.6 Method A |
| 43 | 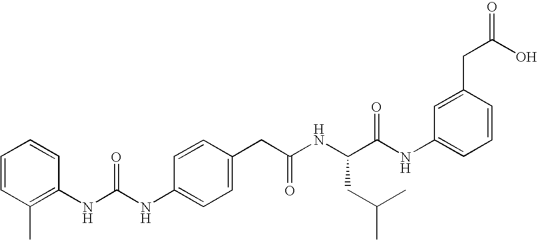 | GP D1; 10 eq. KOH | 83 | white solid | 0.06 (CH₂Cl₂/MeOH/AcOH 9:1:0.1) | 178-181 | 531 [M + H]⁺ | 26.8 Method A |
| 44 | 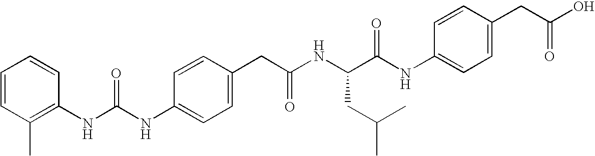 | GP D1; 10 eq. KOH | — | — | — | 206 | — | n.d. |
| 45 | 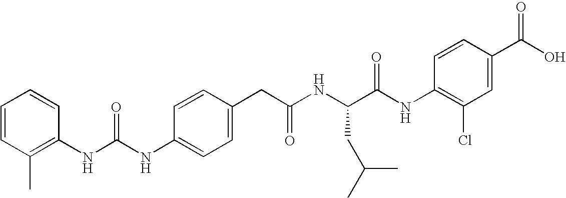 | GP D1; 1.5 eq. KOH | 64 | pale brown solid | 0.34 (CH₂Cl₂/MeOH/AcOH 9:1:0.1) | 154-158 | 551.3 [M + H]⁺ | 22.2 Method B |
| 46 | 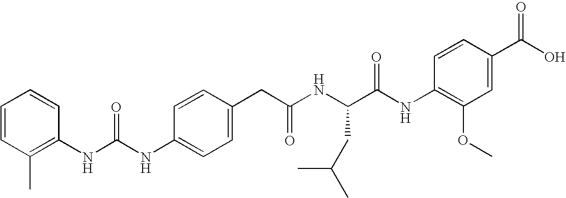 | GP D1; 1.6 eq. NaOH | 16 | white solid | 0.28 (CH₂Cl₂/MeOH/AcOH 9:1:0.1) | 138-142 | 546.8 [M + H]⁺ | 23.1 Method B |
| 47 | 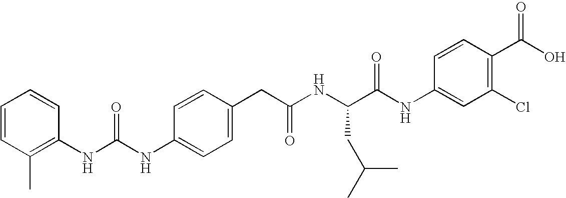 | GP D1; 1.1 eq. KOH | 36 | pale red solid | 0.36 (CH₂Cl₂/MeOH/AcOH 9:1:0.1) | 178-180 | 551.1 [M + H]⁺ | 21.6 Method A |
| 48 | 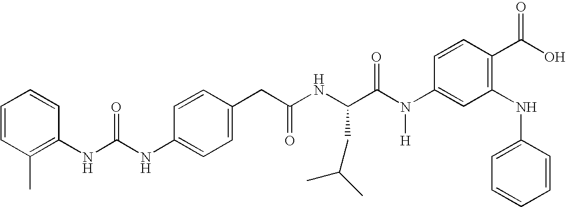 | GP D1; 3.5 eq. KOH | 68 | pale brown solid | 0.30 (CH₂Cl₂/MeOH/AcOH 9:1:0.1) | 164-169 | 608.0 [M + H]⁺ | 23.7 Method A |
| 49 | 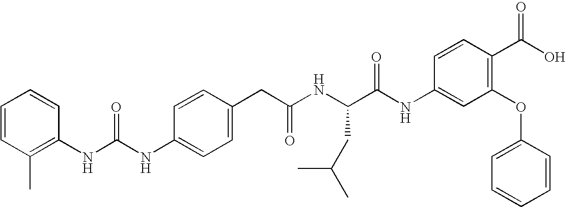 | GP D1; 1.1 eq. KOH | 18 | pale brown solid | 0.62 (CH₂Cl₂/MeOH/AcOH 9:1:0.1) | 158-160 | 609.2 [M + H]⁺ | 23.1 Method A |
| 50 | 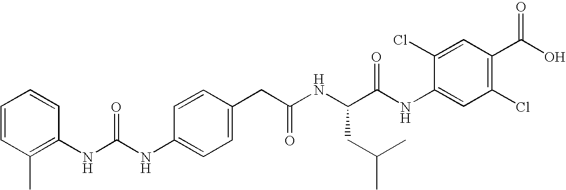 | GP D1; 1.1 eq. KOH | 8 | pale red solid | 0.68 (CH₂Cl₂/MeOH/AcOH 9:1:0.1) | 185-188 | 584.9 [M + H]⁺ | 22.5 Method A |
| 51 | 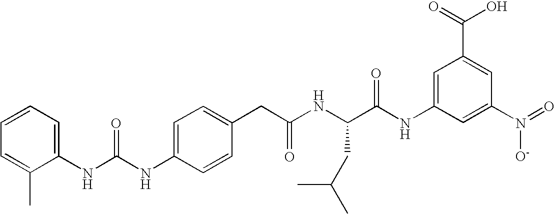 | GP D1; 1.5 eq. NaOH | 45 | yellow solid | 0.10 (CH₂Cl₂/MeOH 9:1) | 188-190 | 562.0 [M + H]⁺ | 23.5 Method A |
| 52 | 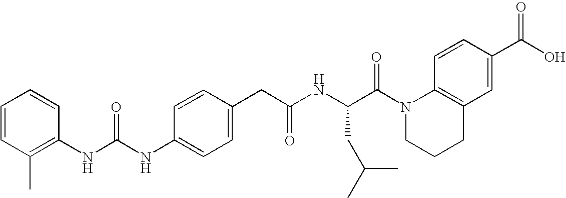 | GP D1; 1.1 eq. KOH | 76 | white foam | n.d. | n.d. | 557 [M + H]⁺HPLC- MS | 22.8 Method A |
| 53 | 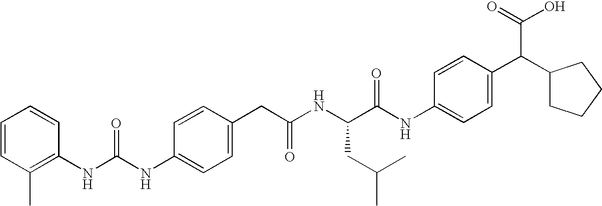 | GP D1; 1.1 eq. KOH | 26 | white solid | 0.74 (CH₂Cl₂/MeOH/AcOH 9:1:0.1) | 206-208 | 599.4 [M + H]⁺ | 19.2 23.5 Method B; 2 dia- steromeres |
| 54 | 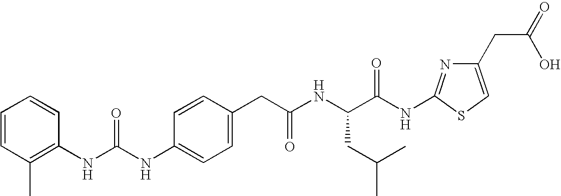 | GP D1; 1.5 eq. KOH | 26 | pale brown solid | 0.30 (CH₂Cl₂/MeOH/AcOH 9:1:0.1) | 178-180 | 538.2 [M + H]⁺ | 29.4 Method A |
| 55 | 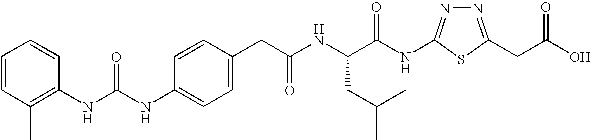 | GP D1; 1.5 eq. KOH | 33 | pale brown solid | 0.18 (CH₂Cl₂/MeOH/AcOH 9:1:0.1) | 187-189 | 539.2 [M + H]⁺ | 28.7 Method A |
| 56 | 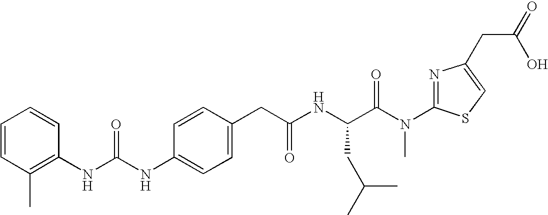 | GP D1; 1.1 eq. KOH | 2 | pale brown solid | 0.26 (CH₂Cl₂/MeOH/AcOH 9:1:0.1) | 172-174 | 552.07 [M + H]⁺ | 21.3 Method A |
| 57 | 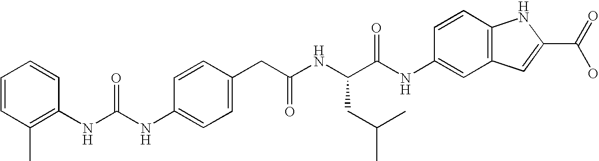 | GP D1; 1.5 eq. KOH | 22 | pale brown solid | 0.08 (CH₂Cl₂/MeOH/AcOH 9:1:0.1) | 240 de- composi- tion | 556.2 [M + H]⁺ | 29.4 Method A |
| 58 | 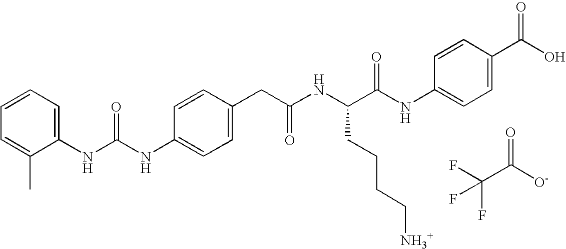 | GP D2 | 3 | white solid | 0.05 (CH₂Cl₂/MeOH/AcOH 9:1:0.1) | 168-169 | 532.1 [M + H]⁺LC-MS | n.d. |
| 59 | 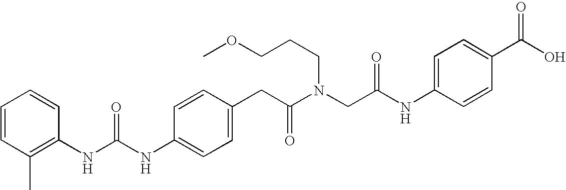 | GP D1; 1.1 eq. KOH | 76 | white solid | 0.48 (CH₂Cl₂/MeOH/AcOH 9:1:0.1) | 210-218 | 570.8 [M + K]⁺ | 20.0 Method A |
| 60 | 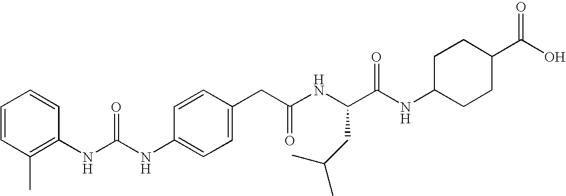 | GP D3 | 95 | white solid | 0.12 (CH₂Cl₂/MeOH/AcOH 9.5:0.5:0.1) | 175 decompo- sition | 523.2 [M + H]⁺ | 20.2 Method B |
| 61 | 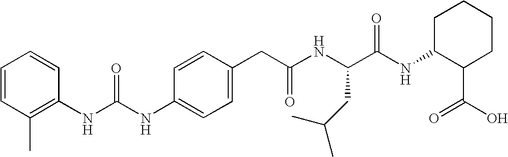 | GP D1, 1.5 eq. KOH | 1 | pale brown solid | 0.10 (CH₂Cl₂/MeOH/AcOH 9.5:0.5:0.1) | n.d. | 523.2 [M + H]⁺ | 20.0 Method A |

In Vitro Assay: Adhesion of Jurkat/Ramos Cells to Immobilized VCAM-1 (Domains 1-3)

Preparation of VCAM-1 (Extracellular Domains 1-3)

Complementary DNA (cDNA) encoding 7 domain form of VCAM-1 (GenBank accession #M60335) was obtained using Rapid-Screen™ cDNA library panels (OriGene Technologies, Inc) at Takara Gene Analysis Center (Shiga, Japan). The primers used were 5′-CCA AGG CAG AGT ACG CAA AC-3′ (sense) and 5′-TGG CAG GTA TTA TTA AGG AG-3′ (antisense). PCR amplification of the 3 domain VCAM-1 cDNA was perform using Pfu DNA polymerase (Stratagene) with the following sets of primers: (U-VCAMd1-3) 5′-CCA TAT GGT ACC TGA TCA ATT TAA AAT CGA GAC CAC CCC AGA A-3′; (L-VCAMd1-3) 5′-CCA TAT AGC AAT CCT AGG TCC AGG GGA GAT CTC AAC AGT AAA-3′. PCR cycle was 94° C. for 45 sec. 55° C. for 45 sec, 72° C. for 2 min, repeating 15 cycles. After the purification of the PCR product, the fragment was digested with KpnI-AvrII. The digested fragment was ligated into pBluescript IISK(−) (Strategene), which was linearized by digesting with KpnI-XhoI. The ligation was followed by transformation to a Dam/Dcm methylase-free E. coli strain SCS110 (Strategene) to create the donor plasmid pHH7. To direct VCAM-1 molecule into the insect cell secretory pathway, the VCAM-1 coding sequence was fused to signal peptide sequence of honeybee melittin. The resulting melittin-VCAM fusion was placed in correct orientation to the baculovirus polyhedrin promoter. Baculovirus transfer vector containing first 3-domain form VCAM-1 (pH 10) was constructed by ligation of 0.9 kb fragment from AvrII/Klenow/BclI digests of pH 7 into SalI/Klenow/BamHI digests of pMelBacB Invitrogen). Recombinant baculovirus was generated by using Bac-N-Blue™ Transfection kit (Invitrogen) according to the manufacture's instruction. The recombinant virus was amplified by infection to High-Five™ insect cells for 5-6 days, and virus titer was determined by plaque assay.

High-Five™ insect cells were pelleted in a 225 ml conical tube by centrifugation at 1000 rpm for 5 min. After discarding the supernatant, the pellet was resuspended in 1.5×10⁹ pfu (MOI=5) of high-titer virus solution, followed by incubation for 1.5 hours at room temperature. The cells were pelleted again and washed once in fresh Express Five™ serum free medium. The cells were pelleted again and finally, resuspended in 200 ml of fresh Express Five TM medium, transferred to a 1,000 ml shaker flask, and incubated in a shaker at 27° C., 130 rpm, for 48 hours before the culture supernatant was collected. The purification of 3-domain form of VCAM-1 from the culture supernatant was performed by one-step anion exchange chromatography. Protein concentration was determined by using Coomassie protein assay reagent (Pierce) according to the manufacture's instruction.

Preparation of VCAM-1 Coated Microtiter Plates

Recombinant human VCAM-1 (extracellular domains 1-3) was dissolved at 1.0 μg/1 ml in PBS. Each well of the microtiter plates (Nalge Nunc International, Fluoro-nunc Cert, 437958) was coated with 100 μl of substrate or for background control with buffer alone for 15 hours at 4 C. After discarding the substrate solution, the wells were blocked using 150 μl per well of block solution Kirkegaard Perry Laboratories, 50-61-01) for 90 minutes. The plate was washed with wash buffer containing 24 mM Tris-HCl (pH 7.4), 137 mM NaCl, 27 mM KCl and 2 MM MnCl₂ just before addition of the assay.

In Vitro Assay using Jurkat Cells

Preparation of Fluorescence Labeled Jurkat Cells:

Jurkat cells (American Type Culture Collection, Clone E6-1, ATCC TIB-152) were cultured in RPM 1640 medium (Nikken Bio Medical Laboratory, CM1101) supplemented with 10% fetal bovine serum (Hyclone, A-1119-L), 100 U/ml penicilin (Gibco BRL, 15140-122) and 100 μg/ml streptomycin (Gibco BRL, 15140-122) in a humidified incubator at 37° C. with 5% CO₂.

Jurkat cells were incubated with phosphate balanced solution (PBS, Nissui, 05913) containing 25 μM of 5 (-and -6)-carboxyfluorescein diacetate, succinimidyle ester (CFSE, Dojindo Laboratories, 345-06441) for 20 min at room temperature while gently swirling every 5 min. After centrifugation at 1000 rpm for 5 min, the cell pellet was resuspended with adhesion assay buffer at a cell density of 4×10⁶ cells/ml. The adhesion assay buffer was composed of 24 mM Tris-HCl (pH 7.4), 137 mM NaCl, 27 mM KCl, 4 mM glucose, 0.1% bovine serum albumin (BSA, Sigma, A9647) and 2 mM MnCl₂.

Assay Procedure (Jurkat Cells)

The assay solution containing each test compounds was transferred to the VCAM-1 coated plates. The final concentration of each test compounds was 5 μM, 10 μM or various concentrations ranging from 0.0001 μM to 10 μM using a standard 5-point serial dilution. The assay solution containing the labeled Jurkat cells was transferred to the VCAM-1 coated plates at a cell density of 2×10⁵ cells per well and incubated for 1 hour at 37 C. The non-adherent cells were removed by washing the plates 3 times with wash buffer. The adherent cells were broken by addition of 1% Triton X-100 (Nacalai Tesque, 355-01). Released CFSC was quantified fluorescence measurement in a fluorometer (Wallac, ARVO 1420 multilabel counter).

The adhesion of Jurkat cells to VCAM-1 was analyzed by percent binding calculated by the formula:

100×(FTS−FBG)(FB−FBG)=% binding, where FTB is the total fluorescent intensity from VCAM-1 coated wells without test compound; FBG is the fluorescent intensity from wells lacking VCAM-1 and FTS is the fluorescent intensity from wells containing the test compound of this invention.

In Vitro Assay using Ramos Cells

Preparation of Fluorescence Labeled Ramos Cells:

Ramos cells (American Type Culture Collection, Clone CRL-1596) were cultured in RPMI 1640 medium (Nikken Bio Medical Laboratory, CM1101) supplemented with 10% fetal bovine serum (Hyclone, A-1119-L), 100 U/ml penicilin (Gibco BRL, 15140-122) and 100 μg/ml streptomycin (Gibco BRL, 15140-122) in a humidified incubator at 37° C. with 5% CO₂.

Ramos cells were incubated with phosphate balanced solution (PBS, Nissui, 05913) containing 25 μM of 5 (-and -6-carboxyfluorescein diacetate, succinimidyle ester (CFSE, Dojindo Laboratories, 345-06441) for 20 min at room temperature while gently swirling every 5 min. After centrifugation at 1000 rpm for 5 min, the cell pellet was resuspended with adhesion assay buffer at a cell density of 4×10⁶ cells/ml. The adhesion assay buffer was composed of 24 mM Tris-HCl (pH 7.4), 137 mM NaCl, 27 mM KCl, 4 mM glucose, 0.1% bovine serum albumin (BSA, Sigma, A9647) and 2 mM MnCl₂.

Assay Procedure (Ramos Cells)

The assay solution containing each test compounds or 5 μg/ml anti-CD49d monoclonal antibody (Immunotech, 0764) was transferred to the VCAM-1 coated plates. The final concentration of each test compounds was 5 μM, 10 μM or various concentrations ranging from 0.0001 μM to 10 μM using a standard 5-point serial dilution. The assay solution containing the labeled Ramos cells was transferred to the VCAM-1 coated plates at a cell density of 2×10⁵ cells per well and incubated for 1 hour at 37 C. The non-adherent cells were removed by washing the plates 3 times with wash buffer. The adherent cells were broken by addition of 1% Triton X-100 (Nacalai Tesque, 355-01). Released CFSC was quantified fluorescence measurement in a fluorometer (Wallac, ARVO 1420 multilabel counter).

The adhesion of Ramos cells to VCAM-1 was analyzed by percent binding calculated by the formula:

100×(FTS−FBG)/(FTB−FBG)=% binding, where FTB is the total fluorescent intensity from VCAM-1 coated wells without test compound; FBG is the fluorescent intensity from wells with anti-CD49d monoclonal antibody and FTS is the fluorescent intensity from wells containing the test compound of this invention.

In Vitro Activity:

In the Jurkat—VCAM-1 assay (indicated as Jurkat—VCAM-1) and the Ramos—VCAM-1 (indicated as Ramos—VCAM-1) the observed IC₅₀ value ranges are indicated Table 4.

D>10 μM≧C>2 μM≧B>0.5 μM≧A

TABLE 4

| No | Structure | IC₅₀ | Cell Type |
|---|---|---|---|
| 27 | 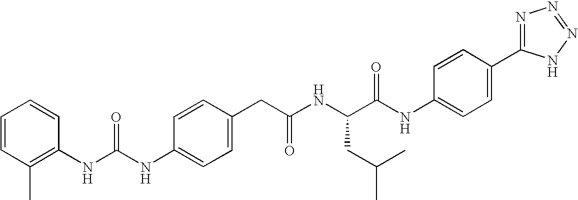 | C | Ramos |
| 39 | 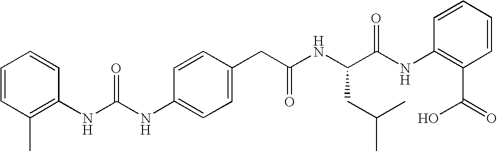 | D | Jurkat |
| 40 | 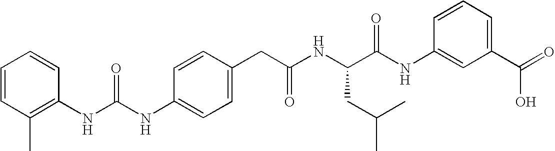 | D | Jurkat |
| 41 | 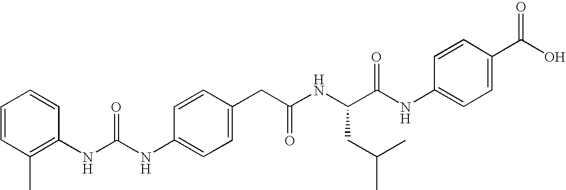 | A | Jurkat |
| 42 | 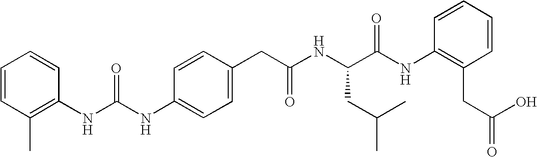 | D | Jurkat |
| 43 | 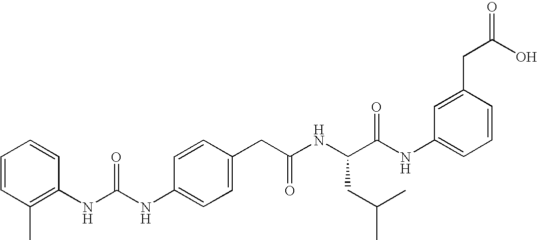 | B | Jurkat |
| 44 | 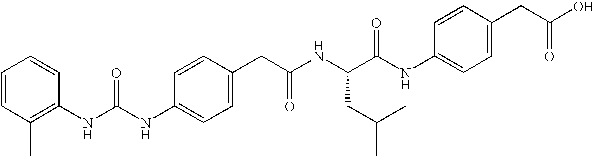 | C | Jurkat |
| 45 | 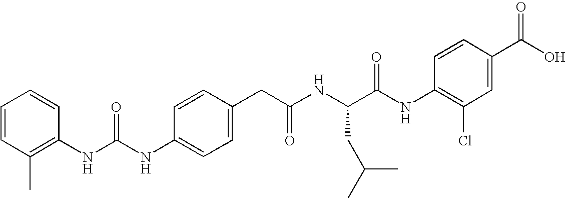 | A-B | Jurkat |
| 46 | 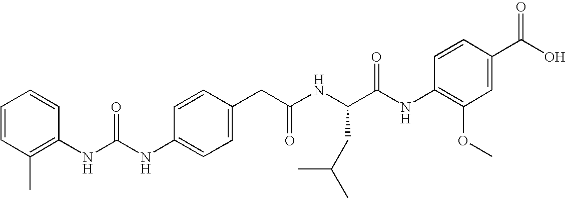 | D | Ramos |
| 47 | 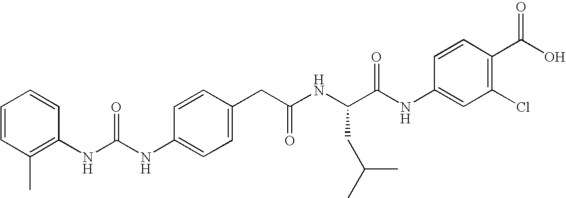 | A | Ramos |
| 48 | 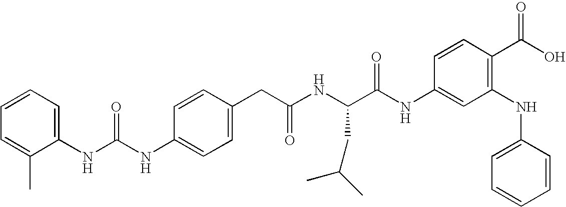 | A | Ramos |
| 49 | 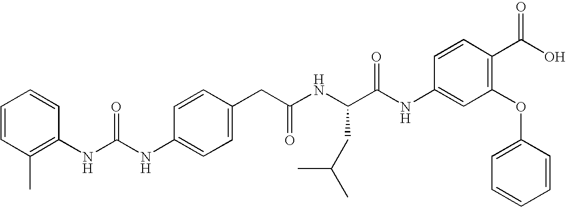 | A | Ramos |
| 50 | 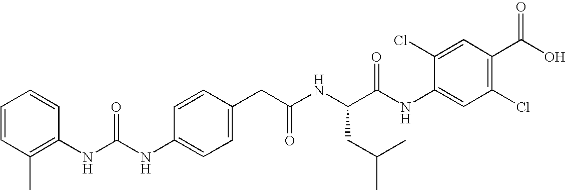 | C | Ramos |
| 51 | 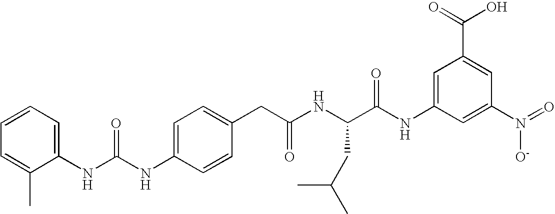 | C | Ramos |
| 52 | 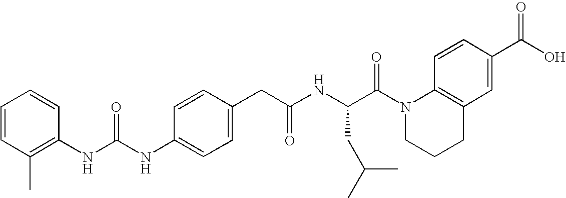 | C | Ramos |
| 53 | 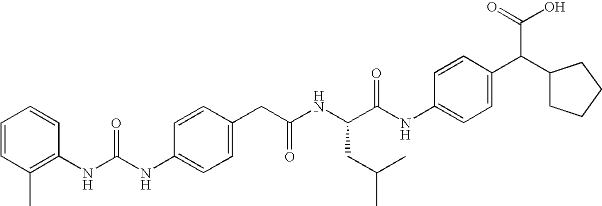 | C | Jurkat |
| 54 | 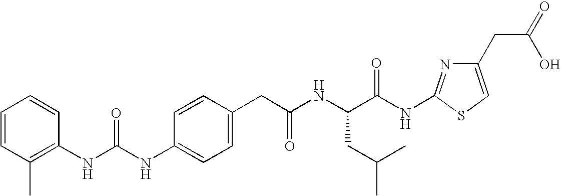 | C | Jurkat |
| 55 | 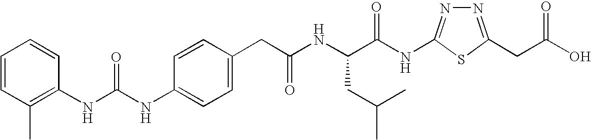 | C | Jurkat |
| 56 | 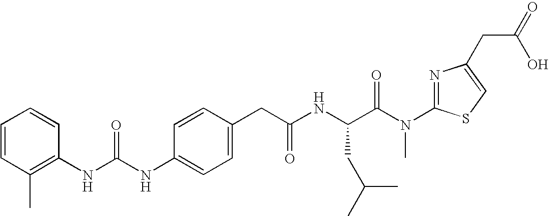 | D | Ramos |
| 57 | 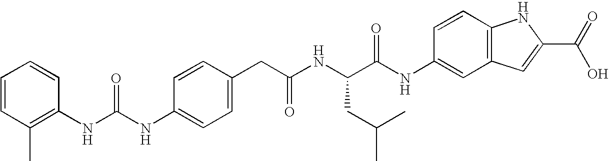 | D | Jurkat |
| 58 | 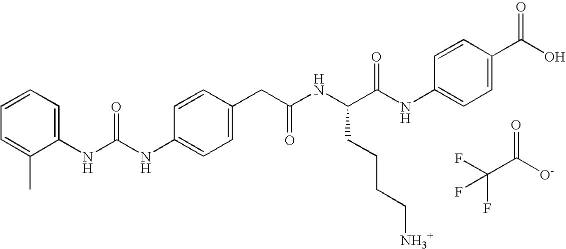 | A | Ramos |
| 59 | 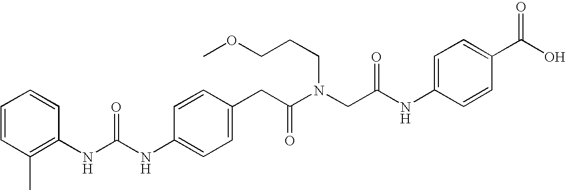 | A | Ramos |
| 60 | 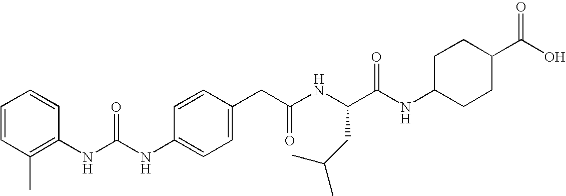 | C | Jurkat |
| 61 | 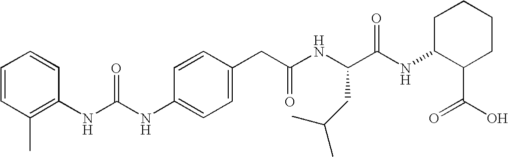 | C | Ramos |

1. Compounds of the general formula (I), 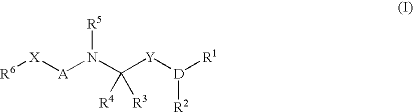
wherein
R¹ represents a 4- to 9-membered saturated, unsaturated or aromatic cyclic residue,
which can contain 0 to 3 heteroatoms selected independently from the group N, S and O,
wherein the cyclic residue R¹ can be annulated with a 4- to 8-membered saturated, unsaturated or aromatic cyclic residue, which can contain 0 to 2 heteroatoms selected independently from the group N, S and O,
and wherein the cyclic residue R¹ and/or a ring annulated to the cyclic residue R¹ is substituted by 1 to 2 substituents —R¹⁻¹—R¹⁻²—R¹⁻³-Z, wherein
R¹⁻¹ represents a bond, —O—, —S—, NR¹⁻⁴, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻¹ can optionally be substituted by 1 to 2 substituents selected from the group R¹⁻⁵,
wherein R¹⁻⁵ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻⁵ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,
R¹⁻² represents a bond, —O—, —S—, NR¹⁻⁴, C₁-C₁₀ alkyl, C₂-C₁₀ alkenyl, C₂-C₁₀ alkynyl,
wherein R¹⁻² can optionally be substituted by C₁-C₁₀ alkyl, C₂-C₁₀ alkenyl, C₂-C₁₀ alkynyl or R¹⁻⁶,
wherein R¹⁻⁶ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻⁶ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,
R¹⁻⁴ can optionally be hydrogen, C₁-C₁₀ alkyl, C₂-C₁₀ alkenyl or C₂-C₁₀ alkynyl,
R¹⁻³ represents a bond, C₁-C₁₀ alkyl, C₂-C₁₀ alkenyl, C₂-C₁₀ alkynyl,
wherein R¹⁻³ can optionally be substituted by C₁-C₁₀ alkyl, C₂-C₁₀alkenyl, C₂-C₁₀ alkynyl or R¹⁻⁷,
wherein R¹⁻⁷ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻⁷ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,
with the proviso that, where R¹⁻³ is a bond, R¹⁻² is not a heteroatom,
and with the proviso that R¹⁻¹ and R¹⁻² are not both heteroatom at the same time,
Z represents —C(O)OR^{Z-1}, —C(O)NR^{Z-2}R^{Z-3}, —SO₂NR^{Z-2}R^{Z-3}, —SO(OR^{Z-1}), —SO₂(OR^{Z-1}), —P(O)R^{Z-1}(OR^{Z-3}), —PO(OR^{Z-1})(OR^{Z-3}) or 5-tetrazolyl,
wherein R^{Z-2} is hydrogen, C₁-C₄ alkyl, C₂-C₆ alkenyl, C₂-C₆ alkynyl, C₃-C₆ cycloalkyl, C₆ or C₁₀ aryl, C(O)R^{Z-4} or —SO₂R^{Z-4},
wherein R^{Z-4} is C₁-C₄ alkyl, C₂-C₆ alkenyl, C₂-C₆ alkynyl, C₃-C₆ cycloalkyl, C₆ or C₁₀ aryl,
wherein R^{Z-4} can optionally be substituted by 1 to 3 substituents selected from the group halogen, nitro, cyano, oxo,
R^{Z-1} and R^{Z-3} are identical or different and represent hydrogen, C₁-C₄ alkyl, C₂-C₆ alkenyl, C₂-C₆ alkynyl, C₃-C₆ cycloalkyl, C₆ or C₁₀ aryl or benzyl,
wherein R^{Z-1} and R^{Z-3} can optionally be substituted by 1 to 3 substituents selected from the group C₁-C₄ alkyl, C₁-C₄ alkyloxy, halogen, nitro, cyano,
the cyclic residue R¹ and/or a ring annulated to the cyclic residue formed by R¹ can optionally be substituted by 0 to 2 substituents R¹⁻⁸, halogen, nitro, amino, cyano and oxo,
wherein
R¹⁻⁸ can independently be selected from the group of C₁-C₄ alkyl, C₁-C₄ alkyloxy, phenyl, phenoxy, phenylamino, C₃-C₆ cycloalkyl, and
R² represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
which can optionally be substituted by 1 to 3 radicals R²⁻¹,
wherein R²⁻¹ represents C₁₋₄ allyl, tifluormethyl, trifluormethoxy, —OR²⁻², —SR²⁻², NR²⁻³R²⁻⁴, —C(O)R²⁻², S(O)R²⁻², —SO₂R²⁻², —CO₂R²⁻², —OC(O)R²⁻², —C(O)NR²⁻³R²⁻⁴, —NR²⁻²C(O)R²⁻³, —SO₂NR²⁻³R²⁻⁴, NR²⁻²SO₂R²⁻³, —NR²⁻²C(O)NR²⁻³R²⁻⁴, —NR²⁻²C(O)OR²⁻³, —OC(O)NR²⁻³R²⁻⁴, halogen, cyano, nitro or oxo,
wherein R²⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R²⁻³ and R²⁻⁴ are identical or different and represent hydrogen, C₁-C₄ alkyl, C₃-C₆ cycloalkyl, C₆ or C₁₀ aryl, or
R²⁻³ and R²⁻⁴ together form a 4-7-membered ring, which includes the nitrogen atom to which R²⁻³ and R²⁻⁴ are bonded and which contains up to 2 additional heteroatoms selected from the group oxygen, nitrogen or sulfur and which contains up to 2 double bonds,
and if R² is alkyl, R² together with the cyclic residue R¹ and D can form a ring,
R³ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
wherein R³ can optionally be substituted by 1 to 3 radicals R³⁻¹,
and wherein R³ can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
which can be annulated with a phenyl ring,
and which can optionally be substituted by 1 to 3 radicals R³⁻¹,
wherein R³⁻¹ represents C₁-C₄ alkyl, trifluormethyl, trifluormethoxy, —OR³⁻², —SR³⁻², NR³⁻³R³⁻⁴, —C(O)R³⁻², S(O)R³⁻², —SO₂R³⁻², —OC(O)R³⁻², —C(O)NR³⁻³R³⁻⁴, —NR³⁻²C(O)R³⁻³, —SO₂NR³⁻³R³⁻⁴, N³⁻²SO₂R³⁻³, —NR³⁻²C(O)NR³⁻³R³⁻⁴, —NR³⁻²C(O)OR³⁻³, —OC(O)NR³⁻³R³⁻⁴, —CO₂R³⁻⁵, halogen, cyano, nitro or oxo,
wherein R³⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R³⁻³ and R³⁻⁴ are identical or different and represent hydrogen, C₁-C₄ alkyl, C₃-C₆ cycloalkyl, C₆ or C₁₀ aryl, benzyl or 9-fluorenylmethyl, or
R³⁻³ and R³⁻⁴ together form a 4-7-membered ring, which includes the nitrogen atom to which R³⁻³ and R³⁻⁴ are bonded and which contains up to 2 additional heteroatoms selected from the group oxygen, nitrogen or sulfur and which contains up to 2 double bonds,
and wherein R³⁻⁵ represents hydrogen, C₁-C₄ alkyl, C₃-C₆ cycloalkyl, C₆ or C₁₀ aryl
R⁴ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
which can optionally be substituted by 1 to 3 radicals R⁴⁻¹,
10 and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
which can optionally be substituted by 1 to 3 radicals R⁴⁻¹,
wherein R⁴⁻¹ represents C₁-C₄ alkyl, trifluormethyl, trifluormethoxy, —OR⁴⁻², —SR⁴⁻², NR⁴⁻³R⁴⁻⁴, —C(O)R⁴⁻², S(O)R⁴⁻², —SO₂R⁴⁻², —OC(O)R⁴⁻², —C(O)NR⁴⁻³R⁴⁻⁴, —NR⁴⁻²C(O)R⁴⁻³, —SO₂NR⁴⁻³R⁴⁻⁴, NR⁴⁻²SO₂R⁴⁻³, —NR⁴⁻²C(O)NR⁴⁻³R⁴⁻⁴, —NR⁴⁻²C(O)OR⁴⁻³, —OC(O)NR⁴⁻³R⁴⁻⁴, —CO₂R⁴⁻⁵, halogen, cyano, nitro or oxo,
wherein R⁴⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R⁴⁻³ and R⁴⁻⁴ are identical or different and represent hydrogen, C₁₋₄ alkyl, C₃-C₆ cycloalkyl, C₆ or C₁₀ aryl, or
R⁴⁻³ and R⁴⁻⁴ together form a 4-7-membered ring, which includes the nitrogen atom to which R⁴⁻³ and R⁴⁻⁴ are bonded and which contains up to 2 additional heteroatoms selected from the group oxygen, nitrogen or sulfur and which contains up to 2 double bonds,
and wherein R⁴⁻⁵ represents hydrogen, C₁-C₄ alkyl, C₃-C₆ cycloalkyl, C₆ or C₁₀ aryl
R⁵ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
which can optionally be substituted by 1 to 3 radicals R⁵⁻¹,
and which can furthermore be single-foldedly substituted by C₃-C₇ cycloalkyl, C₆ or C₁₀ aryl, C₄-C₉ heteroaryl or a saturated or unsaturated heterocyclic residue containing up to 2 heteroatoms selected from the group oxygen, nitrogen or sulfur,
which can optionally be substituted by 1 to 3 radicals R⁵⁻¹,
wherein R⁵⁻¹ represents C₁-C₄ alkyl, phenyl, trifluormethyl, trifluormethoxy, —OR⁵⁻², —SR⁵⁻², NR⁵⁻³R⁵⁻⁴, —C(O)R⁵⁻², S(O)R⁵⁻², —SO₂R⁵⁻², —CO₂R⁵⁻², —OC(O)R⁵⁻², C(O)NR⁵⁻³R⁵⁻⁴, —NR⁵⁻²C(O)R⁵⁻³, —SO₂NR⁵⁻³R⁵⁻⁴, NR⁵⁻²SO₂R⁵⁻³, —NR⁵⁻²C(O)NR⁵⁻³R⁵⁻⁴, —NR⁵⁻²C(O)OR⁵⁻³, —OC(O)NR⁵⁻³R⁵⁻⁴, halogen, cyano, nitro or oxo,
wherein R⁵⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R⁵⁻³ and R⁵⁻⁴ are identical or different and represent hydrogen, C₁-C₄ alkyl, C₃-C₆ cycloalkyl, C₆ or C₁₀ aryl, or
R⁵⁻³ and R⁵⁻⁴ together form a 4-7-membered ring, which includes the nitrogen atom to which R⁵⁻³ and R⁵⁻⁴ are bonded and which contains up to 2 additional heteroatoms selected from the group oxygen, nitrogen or sulfur and which contains up to 2 double bonds,
R⁶ represents phenyl or a 5- to 6-membered aromatic heterocyclic residue containing up to 3 heteroatoms independently selected from the group oxygen, nitrogen or sulfur,
which can optionally be annulated with a 5- to 8-membered saturated or unsaturated cyclic residue containing up to 2 heteroatoms independently selected from the group oxygen, nitrogen or sulfur, and which can optionally be independently substituted by 1 to 3 radicals R⁶⁻¹ and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
wherein the latter cyclic substituents can themselves optionally be substituted by 1 to 3 radicals R⁶⁻¹,
wherein R⁶⁻¹ represents C₁-C₄ alkyl, trifluormethyl, trifluormethoxy, —OR⁶⁻⁴, —SR⁶⁻², NR⁶⁻³R⁶⁻⁴, —C(O)R⁶⁻², S(O)R⁶⁻², —SO₂R⁶⁻², —CO₂R⁶⁻², —OC(O)R⁶⁻², —C(O)NR⁶⁻³R⁶⁻⁴, ——NR⁶⁻²C(O)R⁶⁻², —SO₂NR⁶⁻³R⁶⁻⁴, —NR⁶⁻²SO₂R⁶⁻², —NR⁶⁻²C(O)NR⁶⁻³R⁶⁻⁴, —NR⁶⁻²C(S)NR⁶⁻³R⁶⁻⁴, —NR⁶⁻²C(O)OR⁶⁻⁴, —OC(O)NR⁶⁻³R⁶⁻⁴, halogen, cyano, nitro or oxo,
wherein R⁶⁻² represents hydrogen, C₁-C₄ alkyl, C₃-C₆ cycloalkyl, C₆ or C₁₀ aryl
which can optionally be substituted by 1 to 3 substituents selected from the group C₁-C₄ alkyl, C₁-C₄ alkyloxy, phenyl, C₃-C₆ cycloalkyl, halogen, nitro, cyano,
and wherein R⁶⁻³ and R⁶⁻⁴ are identical or different and represent hydrogen, C₁-C₄ alkyl, C₃-C₆ cycloalkyl, C₆ or C₁₀ aryl or a 4-9-membered saturated or unsaturated heterocyclic residue containing up to 2 heteroatoms selected from the group oxygen, nitrogen or sulfur,
which can optionally be substituted by 1 to 2 substituents selected from the group C₁-C₄ alkyl, phenyl, C₃-C₇ cycloalkyl, C₁-C₄ alkyloxy, halogen, nitro, cyano, or
R⁶⁻³ and R⁶⁻⁴ together form a 4-7-membered ring, which includes the nitrogen atom to which R⁶⁻³ and R⁶⁻⁴ are bonded and which contains up to 2 additional heteroatoms selected from the group oxygen, nitrogen or sulfur and which contains up to 2 double bonds, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo,
and in case that R¹ represents a 3-amino benzoic acid derivative and R⁶⁻¹ represents —OR⁶⁻⁴, —C(O)NR⁶⁻³R⁶⁻⁴ or —NR⁶⁻²C(O)R⁶⁻⁴, then R⁶⁻⁴ represents C₆ or C₁₀ aryl or a 4-9-membered saturated or unsaturated heterocyclic residue containing up to 2 heteroatoms selected from the group oxygen, nitrogen or sulfur,
wherein the ring formed by R⁶⁻³ and R⁶⁻⁴ can optionally be substituted by 1 to 2 substituents selected from the group C₁-C₄ alkyl, phenyl, C₃-C₇ cycloalkyl, C₁-C₄ alkyloxy, halogen, nitro, cyano, or
R² and R³ or R³ and R⁴ or R⁴ and R⁵ together form a 4-7-membered saturated or unsaturated ring containing up to 2 heteroatoms selected from the group oxygen, nitrogen or sulfur, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo and which can be fused with a 3-7 membered homocyclic or heterocyclic, saturated, unsaturated or aromatic ring,
A represents —C(O)—, —C(O)—C(O)—, —SO—, —SO₂—, —PO—, —PO₂—, 2-pyrimidyl, 4-pyrimidyl, 2-pyridyl, 2-imidazolyl, 4-imidazolyl, 2-benzimidazolyl or a ring selected from the following group: 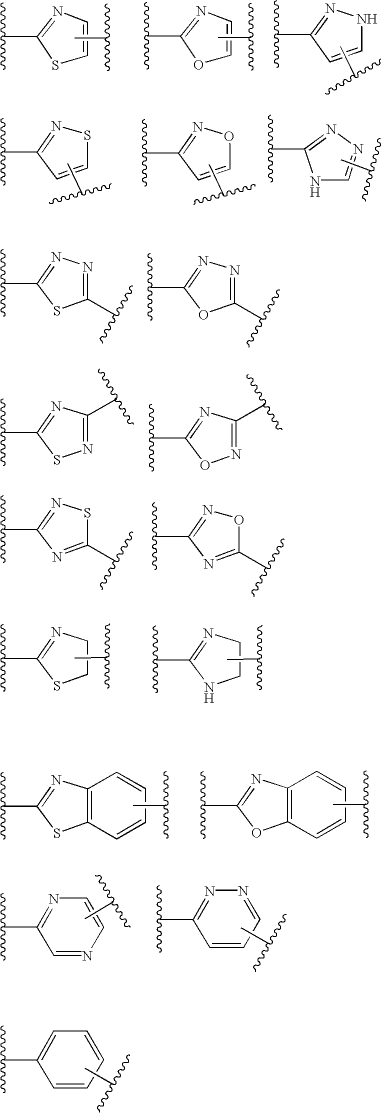
wherein the abovementioned ring systems can optionally be substituted by C₁-C₄ alkyl, C₁-C₄ alkoxy, halogen, nitro, amino, cyano,
X represents —CR^{X-1}R^{X-2},
wherein R^{X-1} and R^{X-2} can be independently selected from the group hydrogen, C₁-C₄ alkyl, C₂-C₄ alkenyl, C₂-C₄ alkynyl, or
together with R⁶ form a 4-7-membered ring, which can contain up to 2 heteroatoms independently selected from the group oxygen, nitrogen or sulfur and containing up to 2 double bonds, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo,
Y represents bond, —C(O)—, —S(O)—, —SO₂—, —O—, —S—, —CR^{Y-1}R^{Y-2}—, or —NR^{Y-3},
wherein R^{Y-1}, R^{Y-2}, R^{Y-3} can be independently selected from the group bond, hydrogen, C₁-C₄ alkyl, C₂-C₄ alkenyl, C₂-C₄ alkynyl,
and can optionally be substituted by 1 to 2 substituents independently selected from the group C₁-C₄ alkyl, phenyl, benzyl, C₃-C₇ cycloalkyl, C₁-C₄ alkyloxy, halogen, nitro, cyano, oxo,
D represents N or CR^{D-1},
wherein R^{D-1} can be independently selected from the group bond, hydrogen, C₁-C₄ alkyl, C₂-C₄ alkenyl, C₂-C₄ alkynyl,
and R^{D-1} can optionally be substituted by 1 to 2 substituents independently selected from the group C₁-C₄ alkyl, phenyl, benzyl, C₃-C₇ cycloalkyl, C₁-C₄ alkyloxy, halogen, nitro, cyano, oxo,
with the proviso that, where D represents —N—, Y does not represent —O— or —S—,
and the compound is not one of the following: 3-[[[(phenylacetyl)amino]acetyl]amino]-benzoic acid; N-(4-aminophenylacetylglycyl)-4-aminophenylacetic acid; N¹-[4-(ethoxycarbonyl)phenyl]-N²-(phenylacetyl)-α-glutamine; N²-benzoyl-N¹-[4-(ethoxycarbonyl)phenyl]-α-glutamine; (S)-4-[[4-carboxy-1-oxo-2-[(phenylacetyl)amino]butyl]amino]-benzeneacetic acid; N-[2-[[4-aminosulfonyl)phenyl]amino]-2-oxoethyl]-N-ethylbenzeneacetamide; N-(2-phenylacetylamino-acetylamino)-benzoic acid ethyl ester,
and pharmaceutically acceptable salts thereof.

2. Compounds of the general formula (I) according to claim 1, 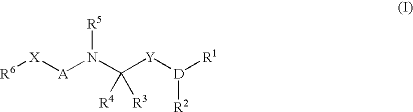
wherein
R¹ represents a 4- to 9-membered saturated, unsaturated or aromatic cyclic residue,
which can contain 0 to 3 heteroatoms selected independently from the group N, S and O,
wherein the cyclic residue R¹ can be annulated with a 4- to 8-membered saturated, unsaturated or aromatic cyclic residue, which can contain 0 to 2 heteroatoms selected independently from the group N, S and O,
and wherein the cyclic residue R¹ and/or a ring annulated to the cyclic residue R¹ is substituted by 1 to 2 substituents —R¹⁻¹—R¹⁻²—R¹⁻³-Z, wherein
R¹⁻¹ represents a bond, —O—, —S—, NR¹⁻⁴, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻¹ can optionally be substituted by 1 to 2 substituents selected from the group R¹⁻⁵,
wherein R¹⁻⁵ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻⁵ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,
R¹⁻² represents a bond, —O—, —S—, NR¹⁻⁴, C₁-C₁₀ alkyl, C₂-C₁₀ alkenyl, C₂-C₁₀ alkynyl,
wherein R¹⁻² can optionally be substituted by C₁-C₁₀ alkyl, C₂-C₁₀ alkenyl, C₂-C₁₀ alkynyl or R¹⁻⁶,
wherein R¹⁻⁶ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻⁶ can optionally be substituted by 1 to 3 substituents selected from the group C₁-C₄ alkyl, C₁-C₄ alkyloxy, phenyl, C₃-C₆ cycloalkyl, halogen, nitro, cyano, oxo,
R¹⁻⁴ can optionally be hydrogen, C₁-C₁₀ alkyl, C₂-C₁₀ alkenyl or C₂-C₁₀ alkynyl,
R¹⁻³ represents a bond, C₁-C₁₀ alkyl, C₂-C₁₀ alkenyl, C₂-C₁₀ alkynyl,
wherein R¹⁻³ can optionally be substituted by C₁-C₁₀ alkyl, C₂-C₁₀ alkenyl, C₂-C₁₀ alkynyl or R¹⁻⁷,
wherein R¹⁻⁷ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 3 heteroatoms selected from the group oxygen, nitrogen or sulfur,
wherein R¹⁻⁷ can optionally be substituted by 1 to 3 substituents selected from the group C₁-C₄ alkyl, C₁-C₄ allyloxy, phenyl, C₃-C₆ cycloalkyl, halogen, nitro, cyano, oxo,
with the proviso that, where R¹⁻³ is a bond, R¹⁻² is not a heteroatom,
and with the proviso that R¹⁻¹ and R¹⁻² are not both heteroatom at the same time,
Z represents —C(O)OR^{Z-1}, —C(O)NR^{Z-2}R^{Z-3}, —SO₂NR^{Z-2}R^{Z-3}, —SO(OR^{Z-1}), —SO₂(OR^{Z-1}), —P(O)R^{Z-1}(OR^{Z-3}), —PO(OR^{Z-1})(OR^{Z-3}) or 5-tetrazolyl,
wherein R^{Z-2} is hydrogen, C₁-C₄ alkyl, C₂-C₆ alkenyl, C₂-C₆ alkynyl, C₃-C₆ cycloalkyl, C₆ or C₁₀ aryl, —C(O)R^{Z-4} or —SO₂R^{Z-4},
wherein R^{Z-4} is C₁-C₄ alkyl, C₂-C₆ alkenyl, C₂-C₆ alkynyl, C₃-C₆ cycloalkyl, C₆ or C₁₀ aryl,
wherein R^{Z-4} can optionally be substituted by 1 to 3 substituents selected from the group halogen, nitro, cyano, oxo,
R^{Z-1} and R^{Z-3} are identical or different and represent hydrogen, C₁-C₄ alkyl, C₂-C₆ alkenyl, C₂-C₆ alkynyl, C₃-C₆ cycloalkyl, C₆ or C₁₀ aryl or benzyl,
wherein R^{Z-1} and R^{Z-3} can optionally be substituted by 1 to 3 substituents selected from the group C₁-C₄ alkyl, C₁-C₄ alkyloxy, halogen, nitro, cyano,
the cyclic residue R¹ and/or a ring annulated to the cyclic residue formed by R¹ can optionally be substituted by 0 to 2 substituents R¹⁻⁸, halogen, nitro, amino, cyano and oxo,
wherein
R¹⁻⁸ can independently be selected from the group of C₁-C₄ alkyl, C₁-C₄ alkyloxy, phenyl, phenoxy, phenylamino, C₃-C₆ cycloalkyl, and
R² represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
which can optionally be substituted by 1 to 3 radicals R²⁻¹,
wherein R²⁻¹ represents C₁₋₄ alkyl, trifluormethyl, trifluormethoxy, —OR²⁻², —SR²⁻², NR²⁻³R²⁻⁴, —C(O)R²⁻², S(O)R²⁻², —SO₂R²⁻², —CO₂R²⁻², —OC(O)R²⁻², —C(O)NR²⁻³R²⁻⁴, —NR²⁻²C(O)R²⁻³, —SO₂NR²⁻³R²⁻⁴, NR²⁻²SO₂R²⁻³, —NR²⁻²C(O)NR²⁻³R²⁻⁴, —NR²⁻²C(O)OR²⁻³, —OC(O)NR²⁻³R²⁻⁴, halogen, cyano, nitro or oxo,
wherein R²⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R²⁻³ and R²⁻⁴ are identical or different and represent hydrogen, C₁-C₄ alkyl, C₃-C₆ cycloalkyl, C₆ or C₁₀ aryl, or
R²⁻³ and R²⁻⁴ together form a 4-7-membered ring, which includes the nitrogen atom to which R²⁻³ and R²⁻⁴ are bonded and which contains up to 2 additional heteroatoms selected from the group oxygen, nitrogen or sulfur and which contains up to 2 double bonds,
and if R² is alkyl, R² together with the cyclic residue R¹ and D can form a ring,
R³ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
wherein R³ can optionally be substituted by 1 to 3 radicals R³⁻¹,
and wherein R³ can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
which can be annulated with a phenyl ring,
and which can optionally be substituted by 1 to 3 radicals R³⁻¹,
wherein R³⁻¹ represents C₁-C₄ alkyl, trifluormethyl, trifluormethoxy, —OR³⁻², —SR³⁻², NR³⁻³R³⁻⁴, —C(O)R³⁻², S(O)R³⁻², —SO₂R³⁻², —OC(O)R³⁻², —C(O)NR³⁻³R³⁻⁴, —NR³⁻²C(O)R³⁻³, —SO₂NR³⁻³R³⁻⁴, NR³⁻²SO₂R³⁻³, —NR³⁻²C(O)NR³⁻³R³⁻⁴, —NR³⁻²C(O)OR³⁻³, —OC(O)NR³⁻³R³⁻⁴, —CO₂R³⁻⁵, halogen, cyano, nitro or oxo,
wherein R³⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R³⁻³ and R³⁻⁴ are identical or different and represent hydrogen, C₁-C₄ alkyl, C₃-C₆ cycloalkyl, C₆ or C₁₀ aryl, benzyl or 9-fluorenylmethyl, or
R³⁻³ and R³⁻⁴ together form a 4-7-membered ring, which includes the nitrogen atom to which R³⁻³ and R³⁻⁴ are bonded and which contains up to 2 additional heteroatoms selected from the group oxygen, nitrogen or sulfur and which contains up to 2 double bonds,
and wherein R³⁻⁵ represents C₁-C₄ alkyl C₃-C₆ cycloalkyl, C₆ or C₁₀ aryl
R⁴ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
which can optionally be substituted by 1 to 3 radicals R⁴⁻¹,
and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
which can optionally be substituted by 1 to 3 radicals R⁴⁻¹,
wherein R⁴⁻¹ represents C₁-C₄ alkyl, trifluormethyl, trifluormethoxy, —OR⁴⁻², —SR⁴⁻², NR⁴⁻³R⁴⁻⁴, —C(O)R⁴⁻², S(O)R⁴⁻², —SO₂R⁴⁻², —OC(O)R⁴⁻², —C(O)NR⁴⁻³R⁴⁻⁴, —NR⁴⁻²C(O)R⁴⁻³, —SO₂NR⁴⁻³R⁴⁻⁴, NR⁴⁻²SO₂R⁴⁻³, —NR⁴⁻²C(O)NR⁴⁻³R⁴⁻⁴, —NR⁴⁻²C(O)OR⁴⁻³, —OC(O)NR⁴⁻³R⁴⁻⁴, —CO₂R⁴⁻⁵, halogen, cyano, nitro or oxo,
wherein R⁴⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R⁴⁻³ and R⁴⁻⁴ are identical or different and represent hydrogen, C₁₋₄ allyl, C₃-C₆ cycloalkyl, C₆ or C₁₀ aryl, or
R⁴⁻³ and R⁴⁻⁴ together form a 4-7-membered ring, which includes the nitrogen atom to which R⁴⁻³ and R⁴⁻⁴ are bonded and which contains up to 2 additional heteroatoms selected from the group oxygen, nitrogen or sulfur and which contains up to 2 double bonds,
and wherein R⁴⁻⁵ represents C₁-C₄ alkyl, C₃-C₆ cycloalkyl, C₆ or C₁₀ aryl
R⁵ represents hydrogen, C₁-C₁₀ alkyl, C₂-C₁₀ alkenyl, C₂-C₁₀ alkynyl, C₆ or C₁₀ aryl, C₃-C₇ cycloalkyl or a 4-9-membered saturated or unsaturated heterocyclic residue containing up to 2 heteroatoms selected from the group oxygen, nitrogen or sulfur,
which can optionally be substituted by 1 to 3 radicals R⁵⁻¹,
and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
which can optionally be substituted by 1 to 3 radicals R⁵⁻¹,
wherein R⁵⁻¹ represents C₁-C₄ alkyl, trifluormethyl, trifluormethoxy, —OR⁵⁻², —SR⁵⁻², NR⁵⁻³R⁵⁻⁴, —C(O)R⁵⁻², S(O)R⁵⁻², —SO₂R⁵⁻², —CO₂R⁵⁻², —OC(O)R⁵⁻², —C(O)NR⁵⁻³R⁵⁻⁴, —NR⁵⁻²C(O)R⁵⁻³, —SO₂NR⁵⁻³R⁵⁻⁴, NR⁵⁻²SO₂R⁵⁻³, —NR⁵⁻²C(O)NR⁵⁻³R⁵⁻⁴, —NR⁵⁻²C(O)OR⁵⁻³, —OC(O)NR⁵⁻³R⁵⁻⁴, halogen, cyano, nitro or oxo,
wherein R⁵⁻² represents hydrogen, C₁-C₄ alkyl, C₃-C₆ cycloalkyl, C₆ or C₁₀ aryl
which can optionally be substituted by 1 substituent selected from the group C₁-C₄ alkyl, C₁-C₄ alkyloxy, phenyl, C₃-C₆ cycloalkyl, halogen, nitro, cyano,
and wherein R⁵⁻³ and R⁵⁻⁴ are identical or different and represent hydrogen, C₁-C₄ alkyl, C₃-C₆ cycloalkyl, C₆ or C₁₀ aryl, or
R⁵⁻³ and R⁵⁻⁴ together form a 4-7-membered ring, which includes the nitrogen atom to which R⁵⁻³ and R⁵⁻⁴ are bonded and which contains up to 2 additional heteroatoms selected from the group oxygen, nitrogen or sulfur and which contains up to 2 double bonds,
R⁶ represents phenyl or a 5- to 6-membered aromatic heterocyclic residue containing up to 3 heteroatoms independently selected from the group oxygen, nitrogen or sulfur,
which can optionally be annulated with a 5- to 8-membered saturated or unsaturated cyclic residue containing up to 2 heteroatoms independently selected from the group oxygen, nitrogen or sulfur,
and which can optionally be independently substituted by 1 to 3 radicals R⁶⁻¹ and which can furthermore be single-foldedly substituted by C₃-C₇ cycloalkyl, C₆ or C₁₀ aryl, C₄-C₉ heteroaryl or a heterocyclic residue containing up to 2 heteroatoms selected from the group oxygen, nitrogen or sulfur,
wherein the latter cyclic substituents can themselves optionally be substituted by 1 to 3 radicals R⁶⁻¹,
wherein R⁶⁻¹ represents C₁-C₄ allyl, trifluormethyl, trifluormethoxy, —OR⁶⁻⁴, —SR⁶⁻², NR⁶⁻³R⁶⁻⁴, —C(O)R⁶⁻², S(O)R⁶⁻², —SO₂R⁶⁻², —CO₂R⁶⁻², OC(O)R⁶⁻², —C(O)NR⁶⁻³R⁶⁻⁴, —NR⁶⁻²C(O)R⁶⁻², —SO₂NR⁶⁻³R⁶⁻⁴, —NR⁶⁻²SO₂R⁶⁻², —NR⁶⁻²C(O)NR⁶⁻³R⁶⁻⁴, —NR⁶⁻²C(O)OR⁶⁻⁴, —OC(O)NR⁶⁻³R⁶⁻⁴, halogen, cyano, nitro or oxo,
wherein R⁶⁻² represents hydrogen, C₁-C₄ alkyl, C₃-C₆ cycloalkyl, C₆ or C₁₀ aryl
which can optionally be substituted by 1 to 3 substituents selected from the group C₁-C₄ alkyl, C₁-C₄ alkyloxy, phenyl, C₃-C₆ cycloalkyl, halogen, nitro, cyano,
and wherein R⁶⁻³ and R⁶⁻⁴ are identical or different and represent hydrogen, C₁-C₄ alkyl, C₃-C₆ cycloalkyl, C₆ or C₁₀ aryl or a 4-9-membered saturated or unsaturated heterocyclic residue containing up to 2 heteroatoms selected from the group oxygen, nitrogen or sulfur,
which can optionally be substituted by 1 to 2 substituents selected from the group C₁-C₄ alkyl, phenyl, C₃-C₇ cycloalkyl, C₁-C₄ alkyloxy, halogen, nitro, cyano, or
R⁶⁻³ and R⁶⁻⁴ together form a 4-7-membered ring, which includes the nitrogen atom to which R⁶⁻³ and R⁶⁻⁴ are bonded and which contains up to 2 additional heteroatoms selected from the group oxygen, nitrogen or sulfur and which contains up to 2 double bonds, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo,
and in case that R¹ represents a 3-amino benzoic acid derivative and R⁶⁻¹-represents —OR⁶⁻⁴, —C(O)NR⁶⁻³R⁶⁻⁴ or —NR⁶⁻²C(O)R⁶⁻⁴, then R⁶⁻⁴ represents C₆ or C₁₀ aryl or a 4-9-membered saturated or unsaturated heterocyclic residue containing up to 2 heteroatoms selected from the group oxygen, nitrogen or
wherein the ring formed by R⁶⁻³ and R⁶⁻⁴ can optionally be substituted by 1 to 2 substituents selected from the group C₁-C₄ alkyl, phenyl, C₃-C₇ cycloalkyl, C₁-C₄ alkyloxy, halogen, nitro, cyano, or
R³ and R⁴ or R⁴ and R⁵ together form a 4-7-membered saturated or unsaturated ring containing up to 2 heteroatoms selected from the group oxygen, nitrogen or sulfur, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo and which can be fused with a 3-7 membered homocyclic or heterocyclic, saturated, unsaturated or aromatic ring,
A represents —C(O)—, —C(O)—C(O)—, —SO—, —SO₂—, —PO—, —PO₂—, 2-pyrimidyl, 4-pyrimidyl, 2-pyridyl, 2-imidazolyl, 4-imidazolyl, 2-benzimidazolyl or a ring selected from the following group: 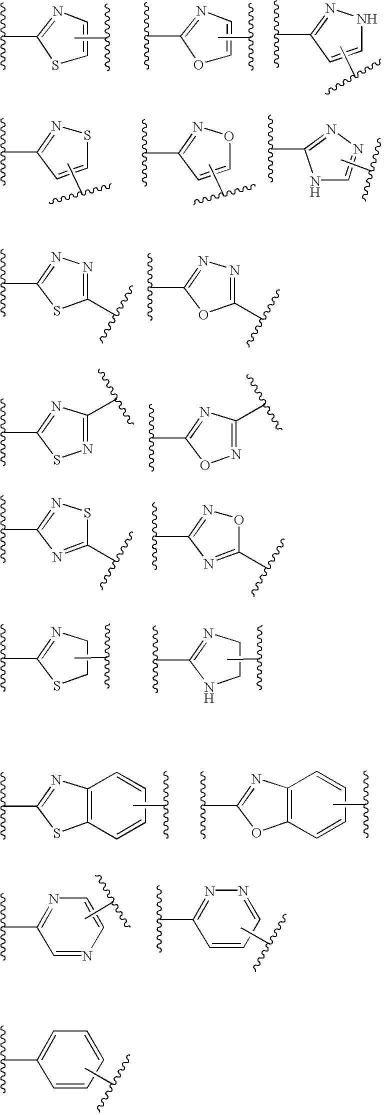
wherein the abovementioned ring systems can optionally be substituted by C₁-C₄ alkyl, C₁-C₄ alkoxy, halogen, nitro, amino, cyano,
X represents —CR^{X-1}R^{X-2},
wherein R^{X-1} and R^{X-2} can be independently selected from the group hydrogen, C₁-C₄ alkyl, C₂-C₄ alkenyl, C₂-C₄ alkynyl, or
together with R⁶ form a 4-7-membered ring, which can contain up to 2 heteroatoms independently selected from the group oxygen, nitrogen or sulfur and containing up to 2 double bonds, which can optionally be substituted by 1 to 2 substituents selected from the group C₁-C₄ alkyl, phenyl, benzyl, C₃-C₇ cycloalkyl, C₁-C₄ alkyloxy, halogen, nitro, cyano, oxo,
Y represents bond, —C(O)—, —S(O)—, —SO₂—, —O—, —S—, —CR^{Y-1}R^{Y-2}—, or —NR^{Y-3},
wherein R^{Y-1}, R^{Y-2}, R^{Y-3} can be independently selected from the group bond, hydrogen, C₁-C₄ alkyl, C₂-C₄ alkenyl, C₂-C₄ alkynyl,
and can optionally be substituted by 1 to 2 substituents independently selected from the group C₁-C₄ alkyl, phenyl, benzyl, C₃-C₇ cycloalkyl, C₁-C₄ alkyloxy, halogen, nitro, cyano, oxo,
D represents N or CR^{D-1},
wherein R^{D-1} can be independently selected from the group bond, hydrogen, C₁-C₄ alkyl, C₂-C₄ alkenyl, C₂-C₄ alkynyl,
and R^{D-1} can optionally be substituted by 1 to 2 substituents independently selected from the group C₁-C₄ alkyl, phenyl, benzyl, C₃-C₇ cycloalkyl, C₁-C₄ alkyloxy, halogen, nitro, cyano, oxo,
with the proviso that, where D represents —N—, Y does not represent —O— or —S—,
and the compound is not one of the following: 3-[[[(phenylacetyl)amino]acetyl]amino]-benzoic acid; N-(4-aminophenylacetylglycyl)-4-aminophenylacetic acid; N¹-[4-(ethoxycarbonyl)phenyl]-N²-(phenylacetyl)-α-glutamine; N²-benzoyl-N¹-[4-(ethoxycarbonyl)phenyl]α-glutamine; (S)-4-[[4-carboxy-1-oxo-2-[(phenylacetyl)amino]butyl]amino]-benzeneacetic acid; N-[2-[[4-aminosulfonyl)phenyl]amino]-2-oxoethyl]-N-ethylbenzeneacetamide; N-(2-phenylacetylamino-acetylamino)-benzoic acid ethyl ester,
and pharmaceutically acceptable salts thereof.

3. Compounds according to claim 1 or 2,
wherein
R¹ represents a 4- to 6-membered saturated, unsaturated or aromatic cyclic residue,
which can contain 0 to 3 heteroatoms selected independently from the group N, S and O,
10 wherein the cyclic residue R¹ can be annulated with a 5- to 6-membered saturated, unsaturated or aromatic cyclic residue, which can contain 0 to 2 heteroatoms selected independently from the group N, S and O,
and wherein the cyclic residue R¹ and/or a ring annulated to the cyclic residue R¹ is substituted by 1 to 2 substituents —R¹⁻¹—R¹⁻²—R¹⁻³-Z, wherein
R¹⁻¹ represents a bond, C₁-C₆ alkyl, C₂-C₆ alkenyl C₂-C₆ alkynyl or C₆ aryl,
wherein R¹⁻¹ can optionally be substituted by 1 substituent selected from the group R¹⁻⁵, wherein R¹⁻⁵ represents hydrogen, C₁-C₆ alkyl, C₂-C₆ alkenyl, C₂-C₆ alkynyl, C₃-C₇ cycloalkyl or C₆ aryl,
R¹⁻² represents a bond, C₁-C₆ alkyl, C₂-C₆ alkenyl, C₂-C₆ alkynyl
R¹⁻³ represents a bond, C₁-C₆ alkyl, C₂-C₆ alkenyl, C₂-C₆ alkynyl
Z represents —C(O)OR^{Z-1}, —C(O)NR^{Z-2}R^{Z-3} or 5-tetrazolyl,
wherein R^{Z-1}, R^{Z-2} and R^{Z-3} are identical or different and represent hydrogen, C₁-C₄ alkyl, C₂-C₆ alkenyl, C₂-C₆ alkynyl or benzyl,
the cyclic residue R¹ and/or a ring annulated to the cyclic residue formed by R¹ can optionally be substituted by 0 to 2 substituents R¹⁻⁸, halogen, nitro, amino, cyano and oxo,
wherein
R¹⁻⁸ can independently be selected from the group of C₁-C₄ alkyl, C₁-C₄ alkyloxy, phenyl, phenoxy, phenylamino,
R² represents hydrogen, C₁-C₆ alkyl, C₂-C₆ alkenyl, C₂-C₆ alkynyl, C₆ aryl, C₅-C₆ cycloalkyl,
and if R² is alkyl, R² together with the cyclic residue R¹ and D can form a 5- to 6-membered ring,
R³ represents hydrogen, C₁-C₆ alkyl, C₂-C₆ alkenyl, C₂-C₆ alkynyl, C₆ aryl, C₅-C₆ cycloalkyl or a 5-6-membered saturated or unsaturated heterocyclic residue containing up to 2 heteroatoms selected from the group oxygen, nitrogen or sulfur,
which can optionally be substituted by 1 radical R³⁻¹,
and wherein R³ can furthermore be single-foldedly substituted by C₃-C₇ cycloalkyl, C₆ aryl, C₄-C₉ heteroaryl or a heterocyclic residue containing up to 2 heteroatoms selected from the group oxygen, nitrogen or sulfur, which can be annulated with a phenyl ring,
wherein R³⁻¹ represents trifluormethyl, trifluormethoxy, —OR³⁻², —SR³⁻², NR³⁻³R³⁻⁴, —NR³⁻²C(O)OR³⁻³, —CO₂R³⁻⁵, halogen, cyano, nitro or oxo,
wherein R³⁻² represents hydrogen or C₁-C₄ alkyl,
and wherein R³⁻³ and R³⁻⁴ are identical or different and represent hydrogen, C₁-C₄ alkyl or benzyl or 9-fluorenylmethyl,
and wherein R³⁻⁵ represents C₁-C₄ alkyl,
R⁴ represents hydrogen, C₁-C₆ alkyl, C₂-C₆ alkenyl, C₂-C₆ or C₆ aryl,
R⁵ represents hydrogen, C₁-C₆ alkyl, C₂-C₆ alkenyl, C₂-C₆ alkynyl or C₆ aryl,
which can optionally be substituted by 1 radical R⁵⁻¹,
wherein R⁵⁻¹ represents trifluormethyl, trifluormethoxy, —OR⁵⁻², —SR⁵⁻², NR⁵⁻³R⁵⁻⁴, halogen, cyano, nitro or oxo,
wherein R⁵⁻², R⁵⁻³ and R⁵⁻⁴ are identical or different and represent hydrogen or C₁-C₄ alkyl
R⁶ represents phenyl or a 5- to 6-membered aromatic heterocyclic residue containing up to 3 heteroatoms independently selected from the group oxygen, nitrogen or sulfur,
and which can optionally be independently substituted by 1 to 3 radicals R⁶⁻¹
wherein R⁶⁻¹ represents —NR⁶⁻²C(O)NR⁶⁻³R⁶⁻⁴,
wherein R⁶⁻² and R⁶⁻³ are identical or different and represent hydrogen or C₁-C₄ alkyl,
and wherein R⁶⁻⁴ represents C₆ aryl,
which can optionally be substituted by 1-2 substituents selected from the group C₁-C₄ alkyl, C₁-C₄ alkyloxy, halogen, nitro, cyano,
or R³ and R⁴ or R⁴ and R⁵ together form a 5-6-membered saturated or unsaturated ring containing up to 2 nitrogen atoms,
A represents —C(O)—, —SO—, —SO₂—,
X represents —CR^{X-1}R^{X-2},
wherein R^{X-1} and R^{X-2} can be independently selected from the group hydrogen, C₁-C₄ alkyl,
Y represents —C(O)—,
D represents —N—,
and pharmaceutically acceptable salts thereof.

4. Compounds according to claim 1, 2 or 3,
wherein
R¹ represents a 5- to 6-membered saturated, unsaturated or aromatic cyclic residue,
which can contain 0 to 3 heteroatoms selected independently from the group N and S,
wherein the cyclic residue R¹ can be annulated with a 5-membered unsaturated or aromatic cyclic residue, which contains 1 nitrogen atom,
and wherein the cyclic residue R¹ and/or a ring annulated to the cyclic residue R¹ is substituted by 1 to 2 substituents —R¹⁻¹—R¹⁻²—R¹⁻³-Z, wherein
R¹⁻¹ represents a bond or Cl alkyl,
wherein R¹⁻¹ can optionally be substituted by cyclopentyl,
R¹⁻² represents a bond,
R¹⁻³ represents a bond,
Z represents —C(O)OR^{Z-1} or 5-tetrazolyl,
R^{Z-1} represents hydrogen, C₁-C₂ alkyl or benzyl,
the cyclic residue R¹ can optionally be substituted by 0 to 2 substituents R¹⁻⁸, halogen and nitro,
wherein
R¹⁻⁸ can independently be selected from the group of C₁-C₄ alkyloxy, phenoxy and phenylamino,
R² represents hydrogen or C₁-C₃ alkyl, or
and if R² is alkyl, R² together with the cyclic residue R¹ and D can form a piperidine ring,
R³ represents hydrogen or C₁-C₄ alkyl,
which can optionally be substituted by 1 radical R³⁻¹,
wherein R³⁻¹ represents NR³⁻³R³⁻⁴ or —NR³⁻²C(O)OR³⁻³,
wherein R³⁻² and R³⁻⁴ represent hydrogen,
R³⁻³ represents hydrogen, benzyl or 9-fluorenylmethyl,
R⁴ represents hydrogen,
R⁵ represents hydrogen or C₃ alkyl,
which can optionally be substituted by 1 radical R⁵⁻¹,
wherein R⁵⁻¹ represents —OR⁵⁻²,
wherein R⁵⁻² represents C₁ alkyl,
R⁶ represents phenyl,
and which is substituted by 1 radical R⁶⁻¹
wherein R⁶⁻¹ represents —NR⁶⁻²C(O)NR⁶⁻³R⁶⁻⁴,
wherein R⁶⁻² represents hydrogen,
and wherein R⁶⁻³ represents hydrogen
and R⁶⁻⁴ represents C₆ aryl,
which is substituted by 1 substituent C₁ alkyl,
A represents —C(O)—,
X represents —CR^{X-1}R^{X-2}—,
wherein R^{X-1} and R^{X-2} represent hydrogen,
Y represents —C(O)—,
D represents N,
and pharmaceutically acceptable salts thereof.

5. Compounds according to claim 1, 2 or 3,
wherein
R¹ represents phenyl,
and wherein the phenyl is substituted by 1 to 2 substituents —R¹⁻¹—R¹⁻²—R¹⁻³-Z,
wherein
R¹⁻¹ represents a bond or C₁ alkyl,
R¹⁻² represents a bond,
R¹⁻³ represents a bond,
Z represents —C(O)OR^{Z-1}
R^{Z-1} represents hydrogen, C₁-C₂ alkyl or benzyl,
R² represents hydrogen,
R³ represents hydrogen, C₁-C₆ alkyl, C₂-C₆ alkenyl, C₂-C₆ alkynyl, C₆ aryl, C₅-C₆ cycloalkyl or a 5-6-membered saturated or unsaturated heterocyclic residue containing up to 2 heteroatoms selected from the group oxygen, nitrogen or sulfur,
which can optionally be substituted by 1 radical R³⁻¹,
and wherein R³ can furthermore be single-foldedly substituted by C₃-C₇ cycloalkyl, C₆ aryl, C₄-C₉ heteroaryl or a heterocyclic residue containing up to 2 heteroatoms selected from the group oxygen, nitrogen or sulfur,
which can be annulated with a phenyl ring,
wherein R³⁻¹ represents trifluormethyl, trifluormethoxy, —OR³⁻², —SR³⁻², —NR³⁻³R³⁻⁴, —NR³⁻²C(O)OR³⁻³, —CO₂R³⁻⁵, halogen, cyano, nitro or oxo,
wherein R³⁻² represents hydrogen or C₁-C₄ alkyl,
and wherein R³⁻³ and R³⁻⁴ are identical or different and represent hydrogen, C₁-C₄ alkyl or benzyl or 9-fluorenylmethyl,
and wherein R³⁻⁵ represents C₁-C₄ alkyl,
R⁴ represents hydrogen,
R⁵ represents hydrogen,
R⁶ represents phenyl,
and which is substituted by 1 radical R⁶⁻¹
wherein R⁶⁻¹ represents —NR⁶⁻²(O)NR⁶⁻³R⁶⁻⁴,
wherein R⁶⁻² represents hydrogen,
and wherein R⁶⁻³ represents hydrogen
and R⁶⁻⁴ represents C₆ aryl,
which is substituted by 1 substituent C₁ alkyl,
or R³ and R⁴ or R⁴ and R⁵ together form a 5-6-membered saturated or a unsaturated ring containing up to 2 nitrogen atoms,
A represents —C(O)—,
X represents —CR^{X-1}R^{X-2},
wherein R^{X-1} and R^{X-2} represent hydrogen,
Y represents —C(O)—,
D represents N,
and pharmaceutically acceptable salts thereof.

6. Compounds according to any one of claims 1 to 5,
wherein
R¹ represents phenyl,
which is 1,4-substituted by a substituent —R¹⁻¹—R¹⁻²—R¹⁻³-Z,
wherein
R¹⁻¹, R¹⁻² and R¹⁻³ represent bonds.

7. Compounds according to any one of claims 1 to 5,
wherein
R¹ represents phenyl,
which is 1,3-substituted by a substituent —R¹⁻¹—R¹⁻²—R¹⁻³-Z,
wherein
R¹⁻¹ represents —CH₂—,
R¹⁻² and R¹⁻³ represent bonds.

8. Compounds according to any one of claims 1 to 5,
wherein
R¹ represents a 5-membered heterocycle.

9. Compounds according to any one of claims 1 to 5,
wherein
R¹ represents a cyclohexyl ring.

10. Compounds according to any one of claims 1 to 5,
wherein R⁶ represents

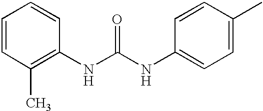

11. Compounds according to any one of claims 1 to 5,
wherein R⁶ represents

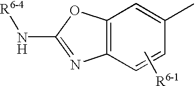

12. Compounds according to claim 1, wherein
R⁵ represents hydrogen, C₁-C₄ alkyl,
which can optionally be substituted by 1 radicals R⁵⁻¹,
and which can furthermore be single-foldedly substituted by C₆ or C₁₀ aryl, C₄-C₉ heteroaryl or a saturated or unsaturated heterocyclic residue containing up to 2 heteroatoms selected from the group oxygen, nitrogen or sulfur,
which can optionally be substituted by 1 to 3 radicals R⁵⁻¹,
wherein R⁵⁻¹ is independently selected from the group C₁-C₄ alkyl, phenyl, trifluormethyl, trifluormethoxy, —OR⁵⁻², NR⁵⁻³R⁵⁻⁴, halogen or oxo,
wherein R⁵⁻² represents hydrogen, C₁-C₄ alkyl, C₃-C₆ cycloalkyl or C₆ aryl
which can optionally be substituted by 1 substituent selected from the group C₁-C₄ alkyl, C₁-C₄ alkyloxy, phenyl, C₃-C₆ cycloalkyl or halogen,
and wherein R⁵⁻³ and R⁵⁻⁴ are identical or different and represent hydrogen, C₁-C₄ alkyl, C₃-C₆ cycloalkyl, C₆ aryl.

13. A process for preparation of compounds of general formula (VII), 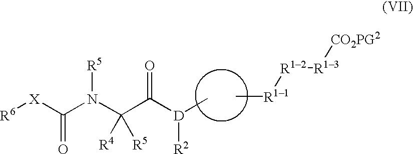
according to any one of claims 5 to 9,
which comprises reaction of carboxylic acids of general formula (V) 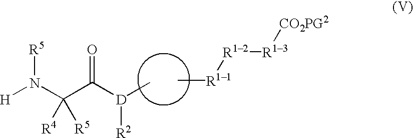
or activated derivatives thereof
with compounds of the general formula (VI) 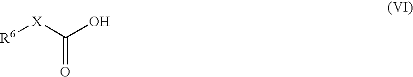
in the presence of a coupling agent and a base in inert solvents.

14. Compounds according to any one of claims 1 to 8, wherein the compound is selected from the following group:
N²-{[4-({[(2-methylphenyl)amino]carbonyl}amino)phenyl]acetyl}-N¹-[4-(1H-tetraazol-5-yl)phenyl]-L-leucinamide,
2-[(N-{[4-({[(2-methylphenyl)amino]carbonyl}amino)phenyl]acetyl}-L-leucyl)amino]benzoic acid,
3-[(N-{[4-({[(2-methylphenyl)amino]carbonyl}amino)phenyl]acetyl}-L-leucyl)amino]benzoic acid,
4-[(N-{[4-({[(2-methylphenyl)amino]carbonyl}amino)phenyl]acetyl}-L-leucyl)amino]benzoic acid,
{2-[(N-{[4-{[(2-methylphenyl)amino]carbonyl}amino)phenyl]acetyl}-L-leucyl)amino]phenyl}acetic acid,
{3-[(N-{[4-({[(2-methylphenyl)amino]carbonyl}amino)phenyl]acetyl}-L-leucyl)amino]phenyl}acetic acid,
{4-[(N-{[4-({[(2-methylphenyl)amino]carbonyl}amino)phenyl]acetyl}-L-leucyl)amino]phenyl}acetic acid,
3-chloro-4-[(N-{[4-({[(2-methylphenyl)amino]carbonyl}amino)phenyl]acetyl}-L-leucyl)amino]benzoic acid,
3-methoxy-4-[(N-{[4-({[(2-methylphenyl)amino]carbonyl}amino)phenyl]acetyl}-L-leucyl)amino]benzoic acid,
2-chloro-4-[(N-{[4-({[(2-methylphenyl)amino]carbonyl}amino)phenyl]acetyl}-L-leucyl)amino]benzoic acid,
2-anilino-4-[(-{[4-({[(2-methylphenyl)amino]carbonyl}amino)phenyl]acetyl}-L-leucyl)amino]benzoic acid,
4-[(N-{[4-({[(2-methylphenyl)amino]carbonyl}amino)phenyl]acetyl}-L-leucyl)amino]-2-phenoxybenzoic acid,
2,5-dichloro-4-[(N-{[4-({[(2-methylphenyl)amino]carbonyl}amino)phenyl]acetyl}-L-leucyl)amino]benzoic acid,
3-[(N-{[4-({[(2-methylphenyl)amino]carbonyl}amino)phenyl]acetyl}-L-leucyl)amino]-5-nitrobenzoic acid,
1-(N-{[4-({[(2-methylphenyl)amino]carbonyl}amino)phenyl]acetyl}-L-leucyl)-1,2,3,4-tetrahydro-6-quinolinecarboxylic acid,
4-{[(N-{[4-({[(2-methylphenyl)amino]carbonyl}amino)phenyl]acetyl}-L-leucyl)amino]methyl}benzoic acid,
cyclopentyl {4-[(N-{[4-({[(2-methylphenyl)amino]carbonyl}amino)phenyl]acetyl}-L-leucyl)amino]phenyl}acetic acid,
{2-[(N-{[4-({[(2-methylphenyl)amino]carbonyl}amino)phenyl]acetyl}-L-leucyl)amino]-1,3-thiazol-4-yl}acetic acid,
{5-[(N-{[4-({[(2-methylphenyl)amino]carbonyl}amino)phenyl]acetyl}-L-leucyl)amino]-1,3,4-thiadiazol-2-yl}acetic acid,
{2-[methyl(N-{[4-({[(2-methylphenyl)amino]carbonyl}amino)phenyl]acetyl}-L-leucyl)amino]-1,3-thiazol-4-yl}acetic acid,
5-[(N-{[4-({[(2-methylphenyl)amino]carbonyl}amino)phenyl]acetyl}-L-leucyl)amino]-1H-indole-2-carboxylic acid
N¹-(4-carboxyphenyl)-N²-{[4-({[(2-methylphenyl)amino]carbonyl}amino)phenyl]acetyl}-L-lysinamide trifluoroacetate,
4-[(N-(3-methoxypropyl)-N-{[4-({[(2-methylphenyl)amino]carbonyl}amino)phenyl]acetyl}glycyl)amino]benzoic acid,
4-[(N-{[4-({[(2-methylphenyl)amino]carbonyl}amino)phenyl]acetyl}-L-leucyl)amino]cyclohexanecarboxylic acid and
(1R,2S)-2-[(N-{[4-({[(2-methylphenyl)amino]carbonyl}amino)phenyl]acetyl}-L-leucyl)amino]cyclohexanecarboxylic acid.

15. The use of a compound according to any one of claims 1 to 5 in the manufacture of a medicament.

16. The use of a compound according to any one of claims 1 to 5 in the manufacture of a medicament for the treatment or the prevention of a condition mediated by integrins.

17. The use of a compound according to any one of claims 1 to 5 in the manufacture of a medicament for the treatment or the prevention of atherosclerosis, asthma, chronic obstructive pulmonary disease (COPD), allergies, diabetes, inflammatory bowel disease, multiple sclerosis, myocardial ischemia, rheumatoid arthritis, transplant rejection and other inflammatory, autoimmune and immune disorders.

18. Pharmaceutical composition, comprising compounds according to any one of claims 1 to 5 and a pharmaceutically acceptable carrier.
